(19) United States
(12) Patent Application Publication
(10) Pub. No.: US 2026/0295264 A1
(43) Pub. Date: Oct. 1, 2026

(54) STIMULATION-EVOKED SIGNAL TYPE DETERMINATION FOR STIMULATION THERAPY

(71) Applicant: MEDTRONIC, INC., Minneapolis, MN (US)

(72) Inventors: Julia P. Slopsema, Minneapolis, MN (US); Hank T. Bink, Golden Valley, MN (US); Abigail L. Skerker, Minneapolis, MN (US); Simeng Zhang, Golden Valley, MN (US); Sarah J. Offutt, Golden Valley, MN (US); Leonid M. Litvak, Bet Shemesh (IL)

(21) Appl. No.: 19/478,640

(22) PCT Filed: Apr. 3, 2024
(86) PCT No.: PCT/IB2024/053241

§ 371 Date: Oct. 24, 2025

Related U.S. Application Data
(60) Provisional application No. 63/498,226, filed on Apr. 25, 2023.

(51) Int. Cl. A61N 1/36; A61N 1/372

(52) U.S. Cl. CPC A61N 1/36139; A61N 1/36007; A61N 1/36171; A61N 1/36175; A61N 1/36185; A61N 1/37235

(57) ABSTRACT

An example method includes determining, for a patient, characteristics of one or more different signal types that are received in response to using at least one setting selected from the group of one or more electrical stimulation settings, one or more sensing settings, or combinations thereof. The example method also includes programming neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

This Application claims priority from U.S. Provisional Patent Application 63/498,226, filed 25 Apr. 2023, the entire content of which is incorporated herein by reference.

### TECHNICAL FIELD

This disclosure generally relates to medical devices, and more specifically, electrical stimulation.

### BACKGROUND

Electrical stimulation devices, sometimes referred to as neurostimulators or neurostimulation devices, may be external to or implanted within a patient, and configured to deliver electrical stimulation therapy to various tissue sites to treat a variety of symptoms or conditions such as chronic pain, tremor, Parkinson's disease, epilepsy, or other neurological disorders, bladder dysfunction such as retention, overactive bladder, urgency, urgency frequency, urinary incontinence, bladder incontinence, bowel incontinence, fecal incontinence, sexual dysfunction, obesity, or gastroparesis. An electrical stimulation device may deliver electrical stimulation therapy via electrodes, e.g., carried by one or more leads, positioned proximate to target locations associated with the brain, the spinal cord, nerves of the pelvis and pelvic floor, tibial nerves, peripheral nerves, the gastrointestinal tract, or elsewhere within a patient. Stimulation proximate the spinal cord, proximate the sacral nerves, within the brain, and proximate peripheral nerves is often referred to as spinal cord stimulation (SCS), sacral neuromodulation (SNM), deep brain stimulation (DBS), and peripheral nerve stimulation (PNS), respectively.

A physician or clinician may select values for a number of programmable stimulation parameters in order to define the electrical stimulation therapy to be delivered by the implantable stimulator to a patient. For example, the physician or clinician may select one or more electrodes, polarities of selected electrodes, a voltage or current amplitude, a pulse width, a pulse frequency, a cycling, and a duration of stimulation as stimulation parameters. A set of therapy stimulation parameters, such as a set including electrode combination or configuration, electrode polarity, amplitude, pulse width, pulse shape, pulse frequency or pulse rate, cycling, or biphasic recharge parameters, or pulse patterns (e.g., masker-probe), may be referred to as a therapy program in the sense that they define the electrical stimulation therapy to be delivered to the patient.

### SUMMARY

A medical device may sense stimulation-evoked signals, and use the stimulation-evoked signals as feedback for controlling the delivery of therapeutic electrical stimulation. However, the characteristics (e.g., content) of the stimulation-evoked signals may be different for different patients (e.g., based on patient physiology or lead placement). For instance, there are different stimulation-evoked signal types, and for different patients, the characteristics of the different stimulation-evoked signal types may indicate which stimulation-evoked signal type is to be used for controlling therapeutic electrical stimulation. The characteristics of the different stimulation-evoked signal types may be due to patient characteristics (e.g., some stimulation-evoked signal types are evoked better than others), due to lead placement and/or electrode type (e.g., some stimulation evoked-evoked signal types are sensed better than others), or due to stimulation parameters (e.g., the characteristics may changes as electrode configuration, electrode polarity, stimulation signal amplitude, frequency, pulse width, pulse shape, pulse rate, cycling,, or biphasic recharge parameters, or pulse patterns, e.g., masker-probe, changes).

This disclosure describes example techniques of determining characteristics of one or more sensed stimulation-evoked signal types in one or more sensed stimulation-evoked signals that are evoked from delivery of electrical stimulation (e.g., by sweeping through stimulation signals meant to evoke the stimulation-evoked signals). In some examples, techniques may include determined characteristics of two or more sensed stimulation-evoked signal types in one or more sensed stimulation-evoked signals that are evoked from delivery of electrical stimulation. A medical device may then utilize the characteristics to determine stimulation signals to evoke subsequent stimulation-evoked signals or device configurations to sense subsequent stimulation-evoked signals. In this manner, the example techniques promote ways in which to more accurately utilize stimulation-evoked signals for determining parameters for therapeutic electrical stimulation.

In one example, this disclosure describes a method including: determining, for a patient, characteristics of one or more different signal types that are received in response to using at least one setting selected from the group of one or more electrical stimulation settings, one or more sensing settings, or combinations thereof, and programming a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

In another example, this disclosure describes a system including: at least one electrode configured to deliver the electrical stimulation to a patient; and a device including processing circuitry configured to: determine, for a patient, characteristics of one or more different signal types that are received in response to using at least two settings selected from the group of one or more electrical stimulation settings, one or more sensing settings, or combinations thereof; and program a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

In another example, this disclosure describes a computer readable medium including instructions that when executed cause one or more processors to: determine, for a patient, characteristics of one or more different signal types that are received in response to using at least two settings selected from the group of one or more electrical stimulation settings, one or more sensing settings, or combinations thereof, and program a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

This summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) in the form of a neurostimulation device configured to deliver sacral neuromodulation (SNM), an external programmer, and one or more sensing devices in accordance with one or more techniques of this disclosure.

FIG. 2 is a block diagram illustrating an example of an IMD in the form of a neurostimulation device, in accordance with one or more techniques of this disclosure.

FIG. 3 is a block diagram illustrating an example of an IMD in the form of a neurostimulation device, in accordance with one or more techniques of this disclosure.

FIG. 4 is a block diagram illustrating an example of an external programmer suitable for use with the IMD of FIG. 2 or 3, in accordance with one or more techniques of this disclosure.

FIG. 5 is a plot of an example stimulation-evoked signal, in accordance with one or more techniques of this disclosure.

FIG. 6 is a plot of another example stimulation-evoked signal, in accordance with one or more techniques of this disclosure.

FIG. 7 is a plot of another example stimulation-evoked signal, in accordance with one or more techniques of this disclosure.

FIG. 8 is a plot of another example stimulation-evoked signal, in accordance with one or more techniques of this disclosure.

FIG. 9 is a plot of an example composite stimulation-evoked signal, in accordance with one or more techniques of this disclosure.

FIG. 10 is a plot of other example composite stimulation-evoked signals, in accordance with one or more techniques of this disclosure.

FIG. 11 is a plot of another example composite stimulation-evoked signal, in accordance with one or more techniques of this disclosure.

FIG. 12 is a flow diagram illustrating an example method of controlling delivery of electrical stimulation therapy, in accordance with one or more techniques of this disclosure.

FIG. 13 is a flow diagram illustrating another example method of controlling delivery of electrical stimulation therapy, in accordance with one or more techniques of this disclosure.

FIG. 14 is a flow diagram illustrating an example method of controlling delivery of electrical stimulation therapy, in accordance with one or more techniques of this disclosure.

FIG. 15 is a flow diagram illustrating an example method of controlling delivery of electrical stimulation therapy, in accordance with one or more techniques of this disclosure.

FIG. 16 is a series of plots of example electrical stimulation pulses according to a masker-probe technique, in accordance with one or more techniques of this disclosure.

FIG. 17 is a series of plots of example sensed stimulation-evoked signals resulting from the pulses of FIG. 16, in accordance with one or more techniques of this disclosure.

### DETAILED DESCRIPTION

Electrical stimulation therapy, e.g., sacral nerve stimulation, tibial nerve stimulation, pudendal nerve stimulation, and/or other types of invasive or noninvasive neuromodulation, may provide bladder dysfunction therapy, pain relief and/or other therapeutic benefits. Electrical stimulation may evoke a response (e.g., a signal) such as a neural response of one or more nerves and contractions of one or more muscles. For example, stimulation of sacral nerves through electrical leads implanted near sacral nerves (e.g., in the third sacral foramen) via sacral neuromodulation may evoke a neural response in adjacent nerves, muscle contractions within the pelvic floor, and distal contractions in the foot. The neural response in nerves and activation/contraction of muscles evoked by electrical stimulation may be captured (e.g., or detected, sensed, measured, and the like) as a stimulation-evoked signal, e.g., via the implanted electrical leads. In some examples, the captured stimulation-evoked signal that may be a composite signal generated from a plurality of signal sources, e.g., a composite of an evoked neural signal, e.g., an evoked compound action potential signal (ECAP) signal, a neural reflex response, or the like, from a nerve, and an electromyography (EMG) signal from one or more muscles.

A stimulation-evoked signal may include one or more features that may indicate and/or guide one or more aspects of electrical stimulation therapy delivery, such as guiding positioning of electrical lead(s) that provides effective therapy, or guiding stimulation programming, or providing feedback in a chronic closed-loop electrical stimulation therapy system. A challenge, however, is that the stimulation parameter settings for delivering electrical stimulation to evoke stimulation-evoked signals, and the sensing settings for sensing stimulation-evoked signals, for different signal types (e.g., ECAP, EMG) may be different. For example, the electrode configuration, recharge, blanking, and techniques for eliciting and sensing an ECAP signal versus EMG signal may be different.

In accordance with one or more techniques of this disclosure, example electrical stimulation systems and example techniques may evoke and sense different stimulation-evoked signal types and determine an operational mode of a medical device based on characteristics of the sensed stimulation-evoked signals (including composite stimulation-evoked signals). For example, an electrical stimulation system may be configured to stimulate and sense ECAP signals, or alternatively to stimulate and sense EMG signals, or to stimulate and sense alternating (e.g., consecutively in time) ECAP and then EMG signals, or to stimulate and sense a composite ECAP and EMG signal, e.g., to concurrently or simultaneously stimulate and sense ECAP and EMG signals.

As an example, a patient that does not have a strong EMG signal may be programmed in ECAP mode to focus sensing parameters to optimally sense ECAP signals. For instance, the characteristics of the EMG signal type of the stimulation-evoked signal may be indicative that the EMG signal type should not be used for controlling therapeutic electrical stimulation signal. Alternatively, a patient with both EMG and ECAP signal types may be programmed using alternating mode to optimally sense EMG and ECAP signals. For instance, the characteristics of the EMG signal type and the ECAP signal type of the stimulation-evoked signal may be indicative that both the EMG signal type and the ECAP signal type should be used for controlling therapeutic electrical stimulation signal.

Systems and techniques disclosed herein provide a number of advantages. For example, disclosed systems and techniques may provide improved electrical stimulation therapy via improving the quantity and quality of determining features indicative of one or more aspects of electrical stimulation therapy. This may be done by improving and/or optimizing sensing of stimulation-evoked signals, which may be tailored to a patient and/or factors particular to a patient.

For example, if a patient, or a particular lead placement within a patient, results in relatively good ECAP signal sensing and relatively poor EMG signal sensing (e.g., the patient is a good “ECAP responder”), the system may tailor electrical stimulation delivery parameter settings and/or sensing settings to optimally evoke and/or sense ECAP signals, e.g., to improve signal quality and feature identification. In some examples, the system may use the ECAP signals to tailor electrical stimulation delivery parameters settings for delivering therapeutic electrical stimulation. Conversely, if a patient, or a particular lead placement within a patient, results in relatively poor ECAP signal sensing and relatively good EMG signal sensing (e.g., the patient is a good “EMG responder”), the system may tailor electrical stimulation delivery parameter settings and/or sensing settings to optimally evoke and/or sense EMG signals, e.g., to improve signal quality and feature identification. In some examples, the system may use the EMG signals to tailor electrical stimulation delivery parameters settings for delivering therapeutic electrical stimulation.

In some examples, if a patient, or a particular lead placement within a patient, results in relatively good ECAP and EMG signals (e.g., the patient is a good “ECAP responder” and a good “EMG responder”), the system may tailor electrical stimulation delivery parameter settings and/or sensing settings to optimally evoke and/or sense ECAP and EMG signals alternatingly (e.g., consecutively) or concurrently (e.g., to sense composite ECAP/EMG signals), e.g., to sense additional and/or different signal types and thereby increase the quantity and/or quality of identification of features from the signals. In some examples, the system may use the ECAP and EMG signals to tailor electrical stimulation delivery parameters settings for delivering therapeutic electrical stimulation.

There may be various ways in which to determine whether the patient is an ECAP responder, EMG responder, or both ECAP responder and EMG responder. As one example, the medical device may be configured to perform a calibration. The calibration may be performed at implantation of the medical device, or periodically as patient physiology can change.

In the calibration, the medical device may sweep through different stimulation parameters for a stimulation signal meant to evoke stimulation-evoked signals, and sweep through different sensing parameters to sense the stimulation-evoked signals. The different stimulation parameters may be different amplitudes, pulse widths, frequencies, electrode configurations. Also, in some examples, for the sensing of the stimulation-evoked signals, the medical device may sense such stimulation-evoked signals on different electrode pairs. Based on the stimulation parameters used and the sensing from different electrode pairs, the medical device, or possibly some other device, may determine how electrical stimulation therapy should be “tuned” (e.g., tuning parameters), for example, to stimulate and sense EMG signals, ECAP signals, both ECAP and EMG signals, or alternating between ECAP and EMG signals.

The medical device may then use the stimulation parameters, determined during the sweep, and the sensing electrode configuration, determined during the sweep, to deliver subsequent stimulation signals to subsequently evoke stimulation-evoked signals (e.g., one or both of the EMG and ECAP based on the type of responder the patient is). The medical device may use the subsequently evoked stimulation-evoked signals as feedback to control the delivery of therapeutic electrical stimulation.

In some examples, the stimulation signals used to evoke stimulation-evoked signals may provide therapeutic benefit, but the techniques are not so limited. In some examples, the therapeutic electrical stimulation may evoke stimulation-evoked signals, but the techniques are not so limited. The medical device may be configured to use the stimulation-evoked signals evoked from the delivery of stimulation signals meant to evoke the stimulation-evoked signals to control the therapeutic electrical stimulation. In some examples, the medical device may be configured to use the stimulation-evoked signals evoked from the delivery of stimulation signals meant to evoke the stimulation-evoked signals and/or evoked from the delivery of the therapeutic electrical stimulation to control the therapeutic electrical stimulation.

In the above examples, EMG and ECAP are described as examples of stimulation-evoked signal types. However, the example techniques should not be considered limited to EMG and ECAP as stimulation-evoked signal types. There may be other examples of stimulation-evoked signal types.

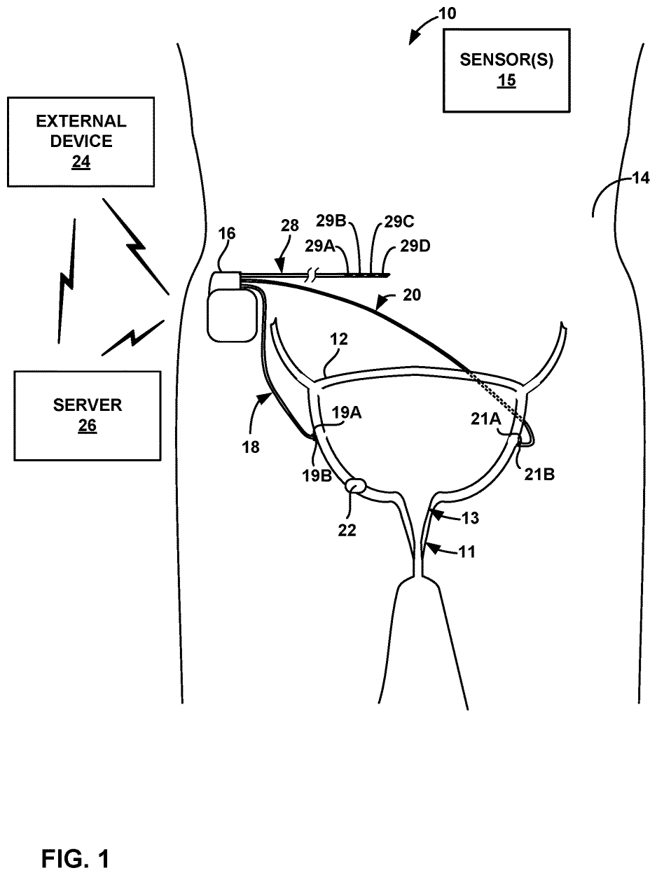

FIG. 1 is a conceptual diagram illustrating an example system 10 that includes an implantable medical device (IMD 16) in the form of a neurostimulation device configured to deliver sacral neuromodulation (SNM), an external programmer, and one or more sensing devices in accordance with one or more techniques of this disclosure. In some examples, system 10 may determine one or more stimulation setting(s) and manage delivery of neurostimulation to patient 14, e.g., to manage bladder dysfunction, such as retention, overactive bladder, urgency, urgency frequency, urinary incontinence, bladder incontinence, bowel incontinence, fecal incontinence.

As shown in the example of FIG. 1, therapy system 10 includes an implantable medical device (IMD) 16 (e.g., an example medical device), which is coupled to leads 18, 20, and 28 and sensor 22. System 10 also includes an external device 24, which is configured to communicate with IMD 16 via wireless communication. System 10 also includes server 26 which may be one or more servers in a cloud computing environment. Server 26 may be configured to communicate with external device 24 and/or IMD 16 via wireless communication through a network access point (not shown in FIG. 1) and may be collocated with external device 24 or may be located elsewhere, such as in a cloud computing data center. IMD 16 generally operates as a therapy device that delivers neurostimulation (e.g., electrical stimulation in the example of FIG. 1) to, for example, a target tissue site proximate a spinal nerve, a sacral nerve, a pudendal nerve, dorsal genital nerve, a tibial nerve, a saphenous nerve, an inferior rectal nerve, a perineal nerve, or other pelvic nerves, branches of any of the aforementioned nerves, roots of any of the aforementioned nerves, ganglia of any of the aforementioned nerves, or plexus of any of the aforementioned nerves. IMD 16 provides electrical stimulation to patient 14 by generating and delivering a programmable electrical stimulation signal (e.g., in the form of electrical pulses or an electrical signal) to a target a therapy site near lead 28 and, more particularly, near electrodes 29A-29D (collectively referred to as “electrodes 29”) disposed proximate to a distal end of lead 28.

IMD 16 may be surgically implanted in patient 14 at any suitable location within patient 14, such as near the pelvis. In some examples, IMD 16 may be implanted in a subcutaneous location in the side of the lower abdomen or the side of the lower back or upper buttocks. IMD 16 has a biocompatible housing, which may be formed from titanium, stainless steel, a liquid crystal polymer, or the like. The proximal ends of leads 18, 20, and 28 are both electrically and mechanically coupled to IMD 16 either directly or indirectly, e.g., via respective lead extensions. Electrical conductors disposed within the lead bodies of leads 18, 20, and 28 electrically connect sense electrodes (e.g., electrodes 19A, 19B, 21A, 21B, 29A, 29B, 29C, and 29D) and stimulation electrodes, such as electrodes 29, to sensing circuitry and a stimulation delivery circuitry (e.g., a stimulation generator) within IMD 16. In the example of FIG. 1, leads 18 and 20 carry electrodes 19A, 19B (collective referred to as “electrodes 19”) and electrodes 21A, 21B (collectively referred to as “electrodes 21”), respectively.

In some examples, external device 24 may collect user input identifying a voiding event, perceived level of fullness, activity, or any other indication of an event associated with the patient. The user input may be in the form of a voiding journal analyzed by external device 24, IMD 16 or server 26, or individual user inputs associated with respective voiding events, leakage, or any other event related to the patient. External device 24 may provide this user input to server 26.

One or more medical leads, e.g., leads 18, 20, and 28, may be connected to IMD 16 and surgically or percutaneously tunneled to place one or more electrodes carried by a distal end of the respective lead at a desired nerve or muscle site, e.g., one of the previously listed target therapy sites such as a tissue site proximate a spinal (e.g., sacral) or pudendal nerve. For example, lead 28 may be positioned such that electrodes 29 deliver electrical stimulation to a spinal, sacral or pudendal nerve to reduce a frequency and/or magnitude of contractions of bladder 12. Additional electrodes of lead 28 and/or electrodes of another lead may provide additional stimulation therapy to other nerves or tissues as well. In FIG. 1, leads 18 and 20 are placed proximate to an exterior surface of the wall of bladder 12 at first and second locations, respectively. In other examples of therapy system 10, IMD 16 may be coupled to more than one lead that includes electrodes for delivery of electrical stimulation to different stimulation sites within patient 14, e.g., to target different nerves.

In the example shown in FIG. 1, leads 18, 20, 28 are cylindrical. Electrodes 19, 21, 29 of leads 18, 20, 28, respectively, may be ring electrodes, segmented electrodes, partial ring electrodes or any suitable electrode configuration. Segmented and partial ring electrodes each extend along an arc less than 360 degrees (e.g., 90-120 degrees) around the outer perimeter of the respective lead 18, 20, 28. In some examples, segmented electrodes 29 of lead 28 may be useful for targeting different fibers of the same or different nerves to generate different physiological effects (e.g., therapeutic effects). In examples, one or more of leads 18, 20, 28 may be, at least in part, paddle-shaped (e.g., a “paddle” lead), and may include an array of electrodes on a common surface, which may or may not be substantially flat.

In some examples, one or more of electrodes 19, 21, 29 may be cuff electrodes that are configured to extend at least partially around a nerve (e.g., extend axially around an outer surface of a nerve). Delivering electrical stimulation via one or more cuff electrodes and/or segmented electrodes may help achieve a more uniform electrical field or activation field distribution relative to the nerve, which may help minimize discomfort to patient 14 that results from the delivery of electrical stimulation. An electrical field may define the volume of tissue that is affected when the electrodes 19, 21, 29 are activated. An activation field represents the neurons that will be activated by the electrical field in the neural tissue proximate to the activated electrodes.

The illustrated numbers and configurations of leads 18, 20, and 28 and electrodes carried by leads 18, 20, and 28 are merely exemplary. Other configurations, e.g., numbers and positions of leads and electrodes are also contemplated. For example, in other implementations, IMD 16 may be coupled to additional leads or lead segments having one or more electrodes positioned at different locations proximate the spinal cord or in the pelvic region of patient 14, and in some examples, IMD 16 may be an electrode, e.g., the outer housing or “can” of IMD 16 may function as an electrode (e.g., to deliver “monopolar” stimulation). The additional leads may be used for delivering different stimulation therapies or other electrical stimulations to respective stimulation sites within patient 14 or for monitoring at least one physiological marker of patient 14.

In some examples, IMD 16 delivers electrical stimulation to at least one of a spinal nerve (e.g., a sacral nerve), a pudendal nerve, dorsal genital nerve, a tibial nerve, a saphenous nerve, an inferior rectal nerve, or a perineal nerve to provide a therapeutic effect that reduces or eliminates a dysfunctional state such as overactive bladder. The desired therapeutic effect may be an inhibitory physiological response related to voiding of patient 14, such as a reduction in bladder contraction frequency by a desired level or degree (e.g., percentage), a reduction in bladder afferent firing, altering a pelvic floor muscle/nerve response and/or status such as of the external urethral sphincter (EUS), levator ani nerve, external anal sphincter, and the like.

A stimulation program may define various parameters of the stimulation signal and electrode configuration which result in a predetermined stimulation intensity being delivered to the targeted nerve or tissue. In some examples, the stimulation program defines parameters for at least one of a current or voltage amplitude of the stimulation signal, a frequency or pulse rate of the stimulation, the shape of the stimulation signal, a duty cycle of the stimulation, a pulse width of the stimulation, a duty cycle of the stimulation ON/OFF periods, biphasic recharge parameters, or pulse patterns (e.g., masker-probe), and/or the combination of electrodes 29 and respective polarities of the subset of electrodes 29 used to deliver the stimulation. Together, these stimulation parameter values (e.g., stimulation parameter settings) may be used to define the stimulation intensity (also referred to herein as a stimulation intensity level). In some examples, if stimulation pulses are delivered in bursts, a burst duty cycle also may contribute to stimulation intensity. Also, independent of intensity, a particular pulse width and/or pulse rate may be selected from a range suitable for causing the desired therapeutic effect after stimulation is terminated and, optionally, during stimulation.

In addition, as described herein, a period during which stimulation is delivered may include on and off periods (e.g., a duty cycle or bursts of pulses) where even the short inter-pulse durations of time when pulses are not delivered are still considered part of the delivery of stimulation. A period during which system 10 withholds stimulation delivery is a period in which no stimulation program is active for IMD 16 (e.g., IMD 16 is not tracking pulse durations or inter-pulse durations that occur as part of the electrical stimulation delivery scheme). In addition to the above stimulation parameters, the stimulation may be defined by other characteristics, such as a time for which stimulation is delivered, a time for which stimulation is terminated, and times during which stimulation is withheld.

System 10 may also include an external device 24, as shown in FIG. 1. External device 24 may be an example of a computing device. In some examples, external device 24 may be a clinician programmer or patient programmer, such as patient programmer 300 described below. In some examples, external device 24 may be a device for inputting information relating to a patient. In some examples, external device 24 may be a wearable communication device, with a therapy request input integrated into a key fob or a wristwatch, handheld computing device, smart phone, computer workstation, or networked computing device. External device 24 may include a user interface that is configured to receive input from a user (e.g., patient 14, a patient caretaker or a clinician). In some examples, the user interface includes, for example, a keypad and a display, which may for example, be a liquid crystal display (LCD) or light emitting diode (LED) display. The keypad may take the form of an alphanumeric keypad or a reduced set of keys associated with particular functions. External device 24 may additionally or alternatively include a peripheral pointing device, such as a mouse, via which a user may interact with the user interface. In some examples, a display of external device 24 may include a touch screen display, and a user may interact with external device 24 via the display. It should be noted that the user may also interact with external device 24, server 26 and/or IMD 16 remotely via a networked computing device.

A user, such as a device manufacturer representative (e.g., sales representative, technical specialist, or the like) a physician, technician, surgeon, electrophysiologist, or other clinician, may also interact with external device 24 or another separate programmer (not shown), such as a clinician programmer, to communicate with IMD 16 and/or server 26. Such a user may interact with external device 24 to retrieve physiological or diagnostic information from IMD 16. The user may also interact with external device 24 to program IMD 16, e.g., select settings, (e.g., alternatively referred to as values) for the stimulation parameter settings (or values) with which IMD 16 generates and delivers stimulation and/or the other operational parameters of IMD 16, such as magnitudes of stimulation energy, user requested periods for stimulation or periods to prevent stimulation, or any other such user customization of therapy. In some examples, the stimulation parameter settings may be proposed by system 10, for example, by IMD 16, and a user may be able to accept or reject the stimulation parameter settings. In other examples, the stimulation parameter settings may be set by system 10, for example, by IMD 16. As discussed herein, the user may also provide input to external device 24 indicative of physiological events such as bladder fill level perception and void events.

In some examples, a healthcare provider may utilize sensor 15, such as wearable sensors or existing implanted sensors, to collect patient data related to sleep, activity or disease symptoms. For example, sensors 15 may be a heartrate sensor or monitor, a blood pressure and/or blood flow sensor, and EMG sensor, a galvanic skin response sensor, an accelerometer, an environmental sensor, such as a microphone, thermometer, hygrometer, a pedometer, a GPS sensor, and/or other sensor to collect patient data, for example, on disease symptoms or lifestyle.

In some examples, the user may use external device 24 to retrieve information from IMD 16 relating to the contraction frequency of bladder 12 and/or voiding events.

As another example, the user may use external device 24 to retrieve information from IMD 16 relating to the performance or integrity of IMD 16 or other components of system 10, such as leads 18, 20, and 28, or a power source of IMD 16. In some examples, this information may be presented to the user as an alert if a system condition that may affect the efficacy of therapy is detected.

The user of external device 24 may also communicate with server 26. For example, the user of external device 24 may provide information relating to the patient to server 26, such as demographic information, medical history, lifestyle information, bladder events, level satisfaction with therapy or sensor data.

Patient 14 may, for example, use a keypad or touch screen of external device 24 to request IMD 16 to deliver or terminate the electrical stimulation, such as when patient 14 senses that a leaking episode may be imminent or when an upcoming void may benefit from terminating therapy that promotes urine retention. In this way, patient 14 may use external device 24 to provide a therapy request to control the delivery of the electrical stimulation “on demand,” e.g., when patient 14 deems the second stimulation therapy desirable. This request may be a therapy trigger event used to terminate electrical stimulation. Patient 14 may also use external device 24 to provide other information to IMD 16, such as information indicative of a phase of a physiological cycle, such as the occurrence of a voiding event.

IMD 16 and external device 24 may communicate via wireless communication using any techniques known in the art. Examples of communication techniques may include, for example, Bluetooth®, but other techniques are also contemplated. In some examples, external device 24 may include a programming lead that may be placed proximate to the patient's body near the IMD 16 implant site in order to improve the quality or security of communication between IMD 16 and external device 24.

IMD 16, in response to commands from external device 24, may deliver electrical stimulation therapy according to a one or more stimulation programs to a target tissue site of the patient 14 via any of electrodes 29A-29D, 19A-19B, and 21A-21B. In some examples, IMD 16 automatically modifies therapy stimulation programs as therapy needs of patient 14 evolve over time. For example, the modification of the therapy stimulation programs may cause the adjustment of at least one parameter of the plurality of stimulation pulses based on received information.

In other examples, electrodes 19 and 21 may be used to detect an electromyogram (EMG) of the detrusor muscle. This EMG may be used to determine the frequency of bladder contractions and the physiological marker of patient 14. The EMG may also be used to detect the strength of the bladder contractions in some examples. As an alternative, or in addition, to an EMG, a strain gauge or other device may be used to detect the status of bladder 12, e.g., by sensing forces indicative of bladder contractions.

In the example of FIG. 1, IMD 16 also may include a sensor 22 for detecting changes in the contraction of bladder 12. Sensor 22 may include, for example, a pressure sensor for detecting changes in bladder pressure, electrodes for sensing pudendal or sacral nerve signals (e.g., afferent and/or efferent), electrodes for sensing urinary sphincter EMG signals (or anal sphincter EMG signals in examples in which system 10 provides therapy to manage fecal urgency or fecal incontinence), or any combination thereof. In examples in which sensor 22 is a pressure sensor, the pressure sensor may be a remote sensor that wirelessly transmits signals to IMD 16 or may be carried on one of leads 18, 20, or 28 or an additional lead coupled to IMD 16. In some examples, IMD 16 may determine whether a contraction frequency of bladder 12 has occurred based on a pressure signal generated by sensor 22. In some examples, IMD 16 may control the timing of the delivery of the electrical stimulation based on input received from sensor 22.

In some examples, IMD 16 and/or external device 24 may be configured to control one or more electrical stimulation parameters based on stimulation-evoked signal(s) and/or composite stimulation-evoked signal(s). For example, IMD 16 and/or external device 24 may be configured to control therapy parameters such as stimulation amplitude, frequency, pulse width, and cycling based on the sensed stimulation-evoked signal(s). In some examples, IMD 16 and/or external device 24 may be configured provide feedback to a user and/or clinician, e.g., via a display screen, and a user and/or clinician may adjust therapy parameters, lead placement and/or positioning, the timing of therapy delivery. In some example, IMD 16 and/or external device 24 may be configured to bypass changes to therapy, e.g., based on a determination that the therapy is effective, e.g., based on composite stimulation-evoked signal(s).

In some examples, one or more stimulation-evoked signal sources (or simply signal sources), such as one or more nerves, one or more muscles, or at least one muscle or at least one nerve, may respond to the electrical stimulation, e.g., via a neural response, a muscle contraction and/or activation, or any other response. In some examples, the response of the one or more sources may be electrical, e.g., an ECAP, an EMG or surface EMG, and the like. In some examples, the response may be mechanical and converted to an electrical signal by a sensor or detector, e.g., by a piezoresistive sensor or other sensor configured to measure muscle contraction and mechanomyography (MMG) and the like. In some examples, nerves may include any of the sacral nerves, e.g., dorsal and ventral rami of sacral nerves, pudendal nerves, sciatic nerves, tibial nerves, saphenous nerves, nerves in the sacral plexus, pelvic nerves, pelvic plexus nerves, pelvic splanchnic nerves, inferior hypogastic plexus nerves, lumbosacral trunk nerves, e.g., where the lumbosacral trunk joins sacral nerves, any sympathetic nerve fibers in the sympathetic chain of any of the above nerves or other nerves. In some examples, one or more muscles may include an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

In some examples, a composite stimulation-evoked signal sensed by one or more sensors and/or electrodes may be a combination of any and/or all of the various signal sources. For example, an electrical stimulation signal may cause a nerve and/or muscle proximate to the stimulation signal to generate a response and other nerves or muscles, not necessarily proximate to the stimulation signal, may also generate responses. The composite stimulation-evoked signal may be a composite of signals from any of the multiple signal sources. In some examples, a composite stimulation-evoked signal may include one or more different stimulation-evoked signal types (e.g., ECAP and EMG are examples of stimulation evoked-signal types). For example, a composite stimulation-evoked signal may include EMG signals from two different sources (a composite of signals of the same type). In other examples, a composite stimulation-evoked signal may include two or more different stimulation-evoked signal types, e.g., an EMG signal and an ECAP signal.

One or more sensors and/or electrodes, such as sensors 15, sensor 22, and/or electrodes 19, 21, and 29, may receive and/or sense signals from the two or more signal sources. In some examples, the received signals may be a composite, e.g., sensors 15, sensor 22, and/or electrodes 19, 21, and 29, may receive and/or sense the signals from one or more signal sources concurrently over a period of time as a single composite stimulation-evoked signal. For example, two or more signals may “arrive” at the sensor (or sensors or electrodes) at the same time and may add together forming the composite signal that is sensed. For example, the two or more signals may be electric signals which may add incoherently, coherently, constructively, destructively, and the like, to form the electric signal that is sensed. In other examples, the two or more signals may be individually sensed and then added and/or combined to form the composite stimulation-evoked signal. For example, electrodes 29 may sense an electric field caused by neural activity of nerve and a sensor 15 may sense an EMG signal caused by a contraction of a muscle, both in response to delivered electrical stimulation. IMD 16 and/or external device 24 may receive each stimulation-evoked signal from two or more sources and then combine the signals to form the composite stimulation-evoked signal.

In some examples, the two or more signal sources may be located relatively far from a sensor/electrode (e.g., sensors 15, sensor 22, and/or electrodes 19, 21, and 29) and/or each other, e.g., at least 1 millimeters (mm) from the sensor and/or electrode and/or each other, at least 10 mm from the sensor and/or electrode and/or each other, at least 100 mm from the sensor and/or electrode and/or each other, at least 200 mm from the sensor and/or electrode and/or each other, at least 1 meter from the sensor and/or electrode and/or each other. For example, two or more signal sources may include a tibial nerve responding to sacral nerve stimulation.

In some examples, the composite stimulation-evoked signal may have a relative long duration, e.g., more than 1 millisecond (ms), more than 5 milliseconds, more than 10 ms, more than 20 ms, etc. For example, because the composite stimulation-evoked signal may originate from multiple signal sources at multiple distances from one or more of the sensors and/or electrodes, and because different signal sources may have different response times, the signals from the signal sources may arrive at, and be captured by, a sensor and/or electrode at different times. In some examples, a sensor and/or electrode may sense signals from signal sources after delivery of every electrical stimulation signal, or a sensor and/or electrode may sense signals from signal sources after an amount of time after delivery of electrical stimulation signals.

As noted above, in some examples, the composite stimulation-evoked signal may comprise signals of different signal types from different signal sources. For example, the composite stimulation-evoked signal may comprise an ECAP signal generated relatively quickly after delivery of electrical stimulation signals, e.g., within about 10 ms, and an EMG signal generated relatively slowly after delivery of electrical stimulation signals, e.g., after about 1 ms. In some examples, the composite stimulation-evoked signal may comprise signals from multiple signal sources that do not overlap in time. For example, the composite stimulation-evoked signal may comprise an ECAP signal from a signal source relatively close to the sensor and/or electrode followed by an EMG signal or another ECAP signal from a different signal source that may be relatively far from the sensor and/or electrode, e.g., such that the ECAP from the close signal source is no longer present while the EMG signal and/or ECAP from the more distant signal source are received by the sensor and/or electrode.

In accordance with one or more aspects of this disclosure, electrodes 19, 21, and 29 and/or sensor(s) 15 may be configured to sense, and IMD 16 and/or external device 24 may be configured to capture, one or more different stimulation-evoked signal types that are received in response to using at least two settings selected from electrical stimulation settings, sensing settings, or combinations thereof. In some examples, electrodes 19, 21, and 29 and/or sensor(s) 15 may be configured to sense, and IMD 16 and/or external device 24 may be configured to capture, two or more different stimulation-evoked signal types that are received in response to using at least two settings selected from electrical stimulation settings, sensing settings, or combinations thereof. For example, electrodes 19, 21, and 29 and/or sensor(s) 15 may be configured to sense an ECAP signal generated by (or evoked from) a first stimulation-evoked signal source, an EMG signal generate by (or evoked from) a second stimulation-evoked signal source different from the first signal source, an EMG signal from the second source following an ECAP signal from the first source, or a composite ECAP and EMG stimulation-evoked signal comprising a composite of ECAP and EMG signals generated by two or more signal sources, e.g., in response to the one or more electrical stimulation signals. In some examples, IMD 16 and/or external device 24 may be configured to control delivery of electrical stimulation signals and/or therapy based on the composite stimulation-evoked signal. For example, IMD 16 and/or external device 24 may be configured to cause one or more electrodes 19, 21, and 29 to deliver one or more electrical stimulation signals to patient 14. In some examples, IMD 16 and/or external device 24 may cause one or more electrodes 19, 21, and 29 to deliver one or more electrical stimulation signals having non-equal pulse amplitudes, non-equal pulse durations, and/or non-equal pulse frequencies. In some examples, IMD 16 and/or external device may be configured to deliver one or more electrical stimulation signals to a sacral nerve (e.g., for SNM therapy), the brain (e.g., DBS therapy), a peripheral nerve (e.g., for PNS and/or PNFS), a saphenous nerve, a tibial nerve, a pudendal nerve, a sciatic nerve, or any other suitable nerve, muscle, and or tissue of patient 14.

Stimulation-evoked signals may be useful to control electrical stimulation therapy. For example, stimulation-evoked signals may be indicative of the efficacy of electrical stimulation therapy, and may provide feedback from which electrical stimulation settings may be verified and/or changed. As described herein, stimulation-evoked signals may be of different types, e.g., originating from different signal sources such as neural sources for ECAPs and muscle sources for EMGs, and may be a composite of a plurality of different stimulation-evoked signal types. For some patients, the content of stimulation-evoked signals and/or signal types evoked by electrical stimulation may be different, e.g., based on patient characteristics, environmental and/or patient posture characteristics, lead placement, or the like.

In some examples, systems and techniques disclosed herein may determine which stimulation-evoked signal types may be best suited, or most efficacious, to evoke and sense, e.g., for a particular patient. Example, stimulation-evoked signal types may be, but are not limited to, an ECAP signal type (typically a faster signal with lower latency and smaller amplitude), and EMG signal type (typically a slow signal with higher latency and a larger amplitude), a composite signal type comprising both an ECAP signal and an EMG signal, which may be consecutive (separated in time) or concurrent (at least partially overlapping in time), a non-ECAP signal type (e.g., a lack of an ECAP signal such as when one may otherwise be expected to be evoked), or a non-EMG signal type (e.g., a lack of an EMG signal such as when one may otherwise be expected to be evoked). A patient, by virtue of patient characteristics, lead placement, environmental characteristics, or the like, may preferentially evoke some signal types or some signal types may be easier to sense, and not others.

To determine a patient response, electrical stimulation may be provided with electrical stimulation settings and sensing settings optimized to generate and sense a particular stimulation-evoked signal type. The subsequently sensed stimulation-evoked signals may comprise the particular stimulation-evoked signal type, or alternatively or additionally, other stimulation-evoked signal types. In some examples, this may optionally be repeated for other signal types, e.g., electrical stimulation may be provided with electrical stimulation settings and sensing settings optimized to generate a different particular stimulation-evoked signal type, and the resulting stimulation-evoked signals may be subsequently sensed. Based on the subsequently sensed stimulation-evoked signals, characteristics of two or more sensed stimulation-evoked signal types may be determined, from which electrical stimulation settings and sensing settings for delivering therapeutic electrical stimulation may be determined.

In accordance with one or more aspects of this disclosure, systems and techniques disclosed herein may enable electrical stimulation to be controlled, improved, and/or optimized based on the content of stimulation-evoked signals and/or stimulation-evoked signal types. In some examples, systems and techniques disclosed herein may enable electrical stimulation to be improved and/or optimized to a particular patient, e.g., based on specific stimulation-evoked signals of the patient. In some examples, IMD 16 and/or external device 24 may be configured to determine and control delivery of electrical stimulation according to an operating mode, e.g., an operating mode of one or more sensors and/or electrodes, such as sensors 15, sensor 22, and/or electrodes 19, 21, and 29.

In some examples, IMD 16 and/or external device 24 may be configured to determine an operating mode, and control delivery of electrical stimulation according to the operating mode, based on stimulation-evoked signals, and the operating mode may be tailored to a particular patient and/or stimulation therapy parameters particular to a patient, e.g., lead placement, environment, or the like. For example, if electrical stimulation does not evoke strong EMG signals from a patient, IMD 16 and/or external device 24 may be configured to operate in an ECAP mode in which electrical stimulation settings and sensing settings may be optimized for evoking and sensing ECAP signals. Conversely, if electrical stimulation does not evoke strong ECAP signals from a patient, IMD 16 and/or external device 24 may be configured to operate in an EMG mode in which electrical stimulation settings and sensing settings may be optimized for evoking and sensing EMG signals. In some examples, if electrical stimulation evokes strong ECAP and EMG signals from a patient, IMD 16 and/or external device 24 may be configured to operate in an alternating mode in which electrical stimulation settings and sensing settings may be optimized for evoking and sensing ECAP signals followed by EMG signals, or a concurrent mode in which electrical stimulation settings and sensing settings may be optimized for evoking and sensing composite ECAP and EMG signals.

In some examples, IMD 16 and/or external device 24 may be configured to execute (or run) a calibration sequence and determine which mode is best suited for a particular patient. For example, IMD 16 and/or external device 24 may execute a calibration sequence in response to a physician calibration request, a patient calibration request, or IMD 16 and/or external device 24 may determine a calibration or calibration update is needed. To calibrate, IMD 16 and/or external device 24 may sweep through electrode configurations (e.g., of electrodes 19, 21, and/or 29), evaluate capture of ECAP, EMG, and/or composite ECAP/EMG stimulation-evoked signals, determine an operating mode based on the captured stimulation-evoked signals and/or one or more features of the captured stimulation-evoked signals, and store stimulation-evoked signals and/or determined stimulation-evoked signal features, e.g., for a plurality of pulse widths.

In some examples, IMD 16 and/or external device 24 may be configured to automatically, or on-demand, update a calibration or recalibrate. For example, IMD 16 and/or external device 24 may be configured to record stimulation-evoked signals and/or signal features at previously selected configurations and/or operating modes and compare new recordings (or capture) of stimulation-evoked signals and/or signal features with the previously recorded (or captured) stimulation-evoked signals and/or signal features. If the new stimulation-evoked signals and/or signal features are within acceptable ranges, IMD 16 and/or external device 24 may be configured to leave electrical stimulation settings and sensing settings the same. If the new stimulation-evoked signals and/or signal features are outside of acceptable ranges (e.g., indicating a change in signals received), IMD 16 and/or external device 24 may be configured to recalibrate and determine new electrical stimulation settings and/or sensing parameters settings and/or a new operating mode.

In some examples, IMD 16 and/or external device 24 may be configured to determine, for a patient, characteristics of two or more sensed stimulation-evoked signal types (e.g., ECAP, EMG) in one or more sensed stimulation-evoked signals (e.g., a single stimulation-evoked signal of a particular stimulation-evoked signal type, consecutive stimulation-evoked signals of different stimulation-evoked signal type, or one or more composite stimulation-evoked signals comprising a plurality of differing stimulation-evoked signal types) that are evoked from delivery of electrical stimulation. IMD 16 and/or external device 24 may be configured to determine, based on the characteristics, at least one of electrical stimulation settings for delivering subsequent electrical stimulation to a patient for evoking one or more subsequent stimulation-evoked signals, or sensing settings for sensing one or more subsequent stimulation-evoked signals from the patient.

IMD 16 and/or external device 24 may be configured to deliver according to the determined electrical stimulation settings, the subsequent electrical stimulation to the patient to evoke the one or more subsequent stimulation-evoked signals from the patient. Additionally or alternatively, IMD 16 and/or external device 24 may be configured to sense, according to the determined sensing settings, the one or more subsequent stimulation-evoked signals from the patient in response to the subsequent electrical stimulation.

In some examples, IMD 16 and/or external device 24 may be configured to determine one or more features of the sensed stimulation-evoked signals, alternating stimulation-evoked signals (e.g., of alternating stimulation-evoked signal types), and/or composite stimulation-evoked signals. For example, IMD 16 and/or external device 24 may be configured to determine a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature. In some examples, IMD 16 and/or external device 24 may be configured to determine an amplitude of one or more peaks of a composite stimulation-evoked signal that are greater than 1 millivolt (mV), or greater than 0.1 mV, or greater than 0.01 mV. In addition, signal might be measured at multiple amplitudes, and the growth curve with one of the above features can be utilized to estimate rate of growth of the signal, or neural threshold, or EMG threshold or inflection point.

Although the example of FIG. 1 is directed to management of bladder dysfunction, in other examples, system 10 may be configured to treat other conditions that may benefit from neurostimulation therapy. For example, system 10 may be used to treat retention, overactive bladder, urgency, urgency frequency, urinary incontinence, bladder incontinence, stress incontinence, nocturia, fecal incontinence, sexual dysfunction, obesity, gastroparesis, intractable constipation, pelvic pain, chronic pain, irritable bowel syndrome, inflammatory bowel disease, interstitial cystitis, neurogenic bowel/bladder, neurological disorders, e.g., tremor, Parkinson's disease, epilepsy, multiple sclerosis, stroke, spinal cord injury, neuropathy, and the like, or psychiatric disorders such as depression, mania, obsessive compulsive disorder, or anxiety disorders. Hence, in some examples, system 10 may be configured to deliver sacral neuromodulation (SNM), sacral neurostimulation (SNS), deep brain stimulation (DBS), peripheral nerve stimulation (PNS), or other stimulation, such as peripheral nerve field stimulation (PNFS), cortical stimulation (CS), gastrointestinal stimulation, or any other stimulation therapy capable of treating a condition of patient 14. In some examples, system 10 may be configured where the electrical stimulation includes stimulation parameters to deliver therapy to address a condition of one or more of painful diabetic neuropathy (PDN), peripheral vascular disease (PVD), peripheral artery disease (PAD), complex regional pain syndrome (CRPS), angina pectoris (AP), leg pain, back pain or pelvic pain.

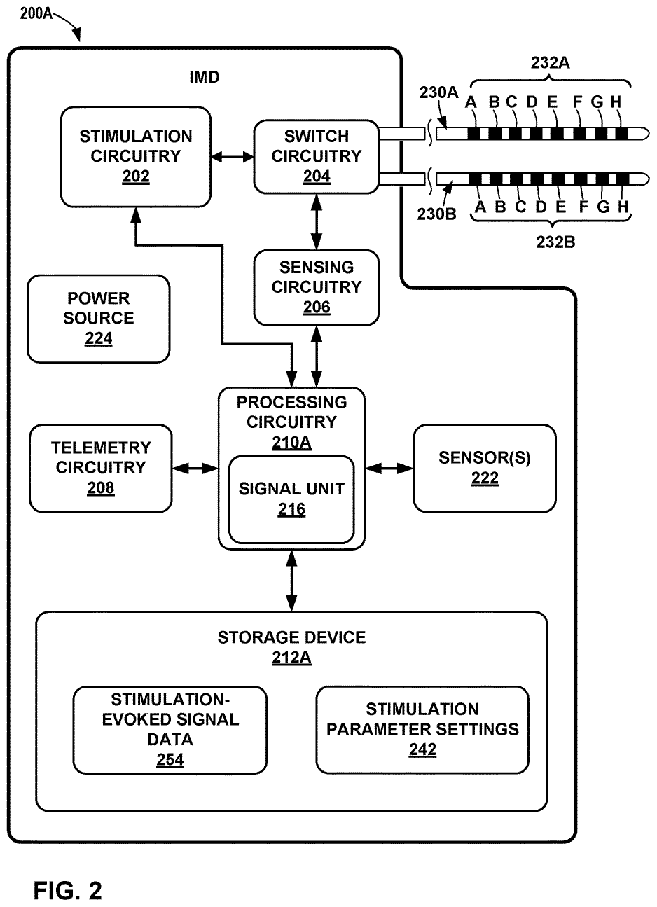

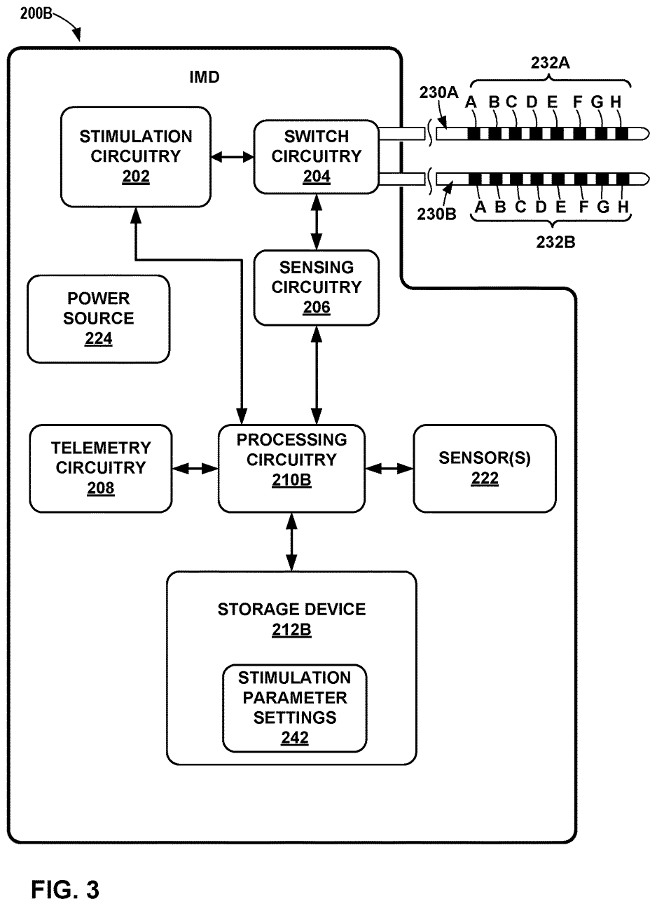

FIGS. 2 and 3 are block diagrams illustrating example configurations of components of an IMD 200A and an IMD 200B, respectively, in accordance with one or more techniques of this disclosure. IMD 200A and/or IMD 200B may be an example of IMD 16 of FIG. 1. In the examples shown in FIGS. 2 and 3, IMD 200A and IMD 200B each include stimulation generation circuitry 202, switch circuitry 204, sensing circuitry 206, telemetry circuitry 208, sensor(s) 222, power source 224, lead 230A carrying electrodes 232A, which may correspond to one of leads 18, 20, 28 and electrodes 19, 21, 29 of FIG. 1, and lead 230B carrying electrodes 232B, which may correspond to another one of leads 18, 20, 28 and electrodes 19, 21, 29 of FIG. 1. In the examples shown in FIG. 2, IMD 200A includes processing circuitry 210A and storage device 212A, and in the example shown in FIG. 3, IMD 200B includes processing circuitry 210B and storage device 212B. Processing circuitry 210A and/or 210B may include one or more processors configured to perform various operations of IMD 200A and/or IMD 200B.

In the examples shown in FIGS. 2 and 3, storage devices 212A and 212B store stimulation parameter settings 242. In addition, as shown in FIG. 2, storage device 212A may store stimulation-evoked signal data 254 obtained directly or indirectly from one or more electrodes 232 and/or sensors 222, or electrodes 19, 21, 29 and/or sensors 15, 22 (FIG. 1). In this case, IMD 200A of FIG. 2 may process stimulation-evoked signal data 254 and select or adjust stimulation parameter settings 242 based on the stimulation-evoked signal data 254.

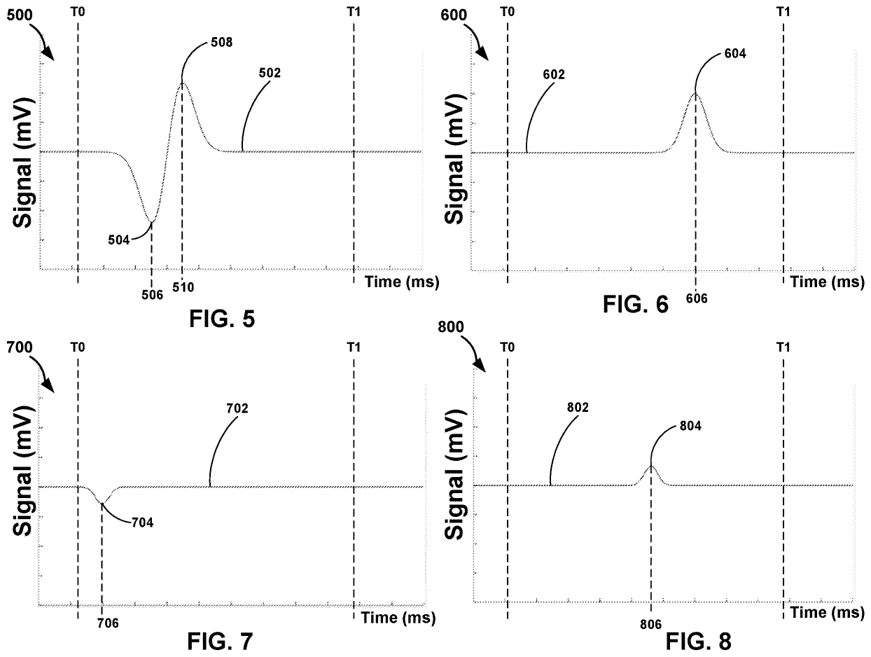

Stimulation-evoked signal data 254 may include sensed signals from one or more signal sources (e.g., which may be stimulation-evoked and referred to as stimulation-evoked signals) and/or sensed composite stimulation-evoked signals, such as those described herein. In some examples, stimulation-evoked signal data 254 may include raw sensed signals from sensor(s) 222 and/or amplified, filtered, and/or analog-to-digital converted signals, e.g., via sensing circuitry 206. For example, stimulation-evoked signal data 254 may include a time-varying signal indicative of a response or responses of one or more signal sources (e.g., nerves and/or muscles) to electrical stimulation, such as illustrated and described below with reference to FIGS. 5-9. In some examples, stimulation-evoked signal data 254 may include an averaged signal and/or one or more signal features determined via processing of the signal, e.g., peak/valley detection, peak/valley amplitude, width, and/or area, frequency analysis, digital signal processing, signal latency, and the like. In some examples, stimulation-evoked signal data 254 may include additional information, such as sensor(s) 222 settings during sensing of stimulation-evoked signals, a timestamp denoting the date and/or time one or more stimulation-evoked signals are sensed, patient information including a current physiological state of patient 14 physiological measurements of patient 14 at or near the time one or more stimulation-evoked signals are sensed, e.g., heart rate, temperature, blood pressure, patient activity, motion, and/or posture (e.g., patient input and/or measured, such as from a patient smartphone, wearable device, external device 24 or 300, or other device) and the like, or patient input such as, voiding and/or voiding frequency, a pain level and/or pain score, patient medical history information, patient age or other demographic information, or any other suitable patient input information.

In one or more examples, such as shown in FIG. 3, the IMD 200B may not store or receive the stimulation-evoked signal data 254. Instead, external device 24 or another device may directly or indirectly select or adjust stimulation parameter settings based on stimulation-evoked signal data 254 and communicate the selected settings or adjustments to IMD 200B of FIG. 3.

In some examples, stimulation parameter settings 242 may include electrical stimulation parameter settings (sometimes referred to as “sets of therapy stimulation parameters”) and/or sensing settings. In some examples, stimulation parameter settings 242 may include respective different stimulation programs selectable by the clinician or patient for therapy, or settings for automatically determining a stimulation program and/or operating mode. In some examples, stimulation parameter settings 242 may include one or more recommended parameter settings. In this manner, each stored therapy stimulation program, or set of stimulation parameters, of stimulation parameter settings 242 defines values for a set of electrical stimulation parameters (e.g., a stimulation parameter set), such as electrode combination (selected electrodes and polarities), stimulation current or voltage amplitude, stimulation pulse width, and pulse frequency.

Stimulation generation circuitry 202 includes electrical stimulation circuitry configured to generate electrical stimulation and generates electrical stimulation pulses selected to alleviate symptoms of one or more diseases, disorders or syndromes. While stimulation pulses are described, stimulation signals may take other forms, such as continuous-time signals (e.g., sine waves) or the like. The electrical stimulation circuitry may reside in an implantable housing, for example of the IMD. Each of leads 230A, 230B may include any number of electrodes 232A, 232B. The electrodes are configured to deliver the electrical stimulation to the patient. In the example of FIGS. 2 and 3, each set of electrodes 232A, 232B includes eight electrodes A-H. In some examples, the electrodes are arranged in monopolar combinations. In some examples, the electrodes are arranged in bipolar combinations and/or tripolar combinations. A bipolar electrode combination or a tripolar electrode combination may use electrodes carried by the same lead 230A, 230B or different leads. For example, an electrode A of electrodes 232A may be a cathode and an electrode B of electrodes 232A may be an anode, forming a bipolar combination. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232A, 232B, or directed sensed signals from one or more of electrodes 232A, 232B to sensing circuitry 206. In some examples, each of the electrodes 232A, 232B may be associated with respective current source and sink circuitry to selectively and independently configure the electrode to be a cathode or anode. Stimulation generation circuitry 202 and/or sensing circuitry 206 also may include sensing circuitry to direct electrical signals sensed at one or more of electrodes 232A, 232B.

Sensing circuitry 206 may be configured to monitor signals from any combination of electrodes 232A, 232B and/or sensor(s) 222. In some examples, sensing circuitry 206 includes one or more amplifiers, filters, averaging, and analog-to-digital converters. Sensing circuitry 206 may be used to sense stimulation-evoked and/or physiological signals, such as ECAP signals, EMG signals, and the like. In some examples, sensing circuitry 206 detects ECAP and/or EMG signals from a particular combination of electrodes 232A, 232B. In some cases, the particular combination of electrodes for sensing ECAP and/or EMG signals includes different electrodes than a set of electrodes 232A, 232B used to deliver stimulation pulses. Alternatively, in other cases, the particular combination of electrodes used for sensing ECAP and/or EMG signals includes at least one of the same electrodes as a set of electrodes used to deliver stimulation pulses to patient 14. Sensing circuitry 206 may provide signals to an analog-to-digital converter, for conversion into a digital signal for processing, analysis, storage, feature extraction, classification, or output by processing circuitry 210. In some examples, sensing circuitry 206 may sense and/or detect stimulation-evoked signals and/or composite stimulation-evoked signals comprising one or more of an ECAP, an EMG or surface EMG, an MMG, a network excitability, and/or multiple signals of differing signal type evoked by one or more signal sources such as sacral nerves, e.g., dorsal and ventral rami of sacral nerves, pudendal nerves, sciatic nerves, tibial nerve, saphenous nerves, nerves in the sacral plexus, pelvic nerves, pelvic plexus nerves, pelvic splanchnic nerves, inferior hypogastic plexus nerves, lumbosacral trunk nerves, e.g., where the lumbosacral trunk joins sacral nerves, any sympathetic nerve fibers in the sympathetic chain of any of the above nerves or other nerves, muscles such as an external anal sphincter muscle, coccygeus muscle, levator ani muscle group, bulbocavernosus and/or bulbospongiosus muscle, gluteal muscles, e.g., gluteal maximus, gluteal medius, and gluteal minimus, perineal muscles, ischiocavernosus muscles, puborectalis muscles, piriformis muscles, or any other muscles.

Sensor(s) 222 may be configured to sense one or more physiological responses of a patient, e.g., patient 14. In some examples, sensor(s) 222 may be substantially the same as sensor(s) 15, 22 described above with reference to FIG. 1. In some examples, sensors 222 may be other sensors located at one or more other positions on patient 14, located at or near one or more muscles and or nerves, or located at positions on patient 14 which may be relatively far from a signal source, e.g., a nerve or muscle.

Telemetry circuitry 208 supports wireless communication between IMD 200A and/or IMD 200B and an external programmer or another computing device under the control of processing circuitry 210. Processing circuitry 210A and/or 210B of IMD 200A and/or IMD 200B, respectively, may receive, as updates to programs, values for various stimulation parameters such as amplitude and electrode combination, from the external programmer via telemetry circuitry 208. Processing circuitry 210A and/or 210B of IMD 200A and/or IMD 200B, respectively, may store updates to the stimulation parameter settings 242 or any other data in storage device 212. Telemetry circuitry 208 in IMD 200A and/or IMD 200B, as well as telemetry circuits in other devices and systems described herein, such as the external programmer and patient feedback sensing system, may accomplish communication by radiofrequency (RF) communication techniques. In addition, telemetry circuitry 208 may communicate with an external medical device programmer via proximal inductive interaction of IMD 200A and/or IMD 200B with the external programmer, where the external programmer may be one example of external device 24 of FIG. 1. Accordingly, telemetry circuitry 208 may send information to the external programmer on a continuous basis, at periodic intervals, or upon request from IMD 16 and/or external device 24.

Processing circuitry 210A and/or 210B may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210A and/or 210B herein may be embodied as firmware, hardware, software or any combination thereof. Processing circuitry 210A and/or 210B controls stimulation generation circuitry 202 to generate stimulation signals according to stimulation parameter settings 242. In some examples, processing circuitry 210A and/or 210B may execute other instructions stored in storage device 212A and/or 212B, respectively, to apply stimulation parameters specified by one or more of programs, such as electrode combination or configuration, electrode polarity, amplitude, pulse width, pulse shape, pulse frequency or pulse rate, cycling, or biphasic recharge parameters, or pulse patterns (e.g., masker-probe) of each of the stimulation signals.

In the illustrated example of FIG. 2, processing circuitry 210A includes a signal unit 216 to process stimulation-evoked signals and/or composite stimulation evoked signals. Signal unit 216 may represent an example of a portion of processing circuitry configured to process stimulation-evoked signals and/or composite stimulation-evoked signals received from a sensor, such as sensor(s) 222 and/or sensor(s) 15, 22, and/or a patient-input device, such as external device 24 or a patient device such as the patient's phone and/or computing device. In the example of FIG. 3, the processing of stimulation-evoked signals and/or composite stimulation-evoked signals occurs in a device other than IMD 200B.

Referring again to FIG. 2, the signal unit 216, discussed further below, receives information regarding stimulation-evoked signals and/or composite stimulation-evoked signals, such as information relating to sensed and/or received stimulation-evoked signals and/or composite stimulation-evoked signals associated with the efficacy of the electrical stimulation therapy, and controls the electrical stimulation circuitry 202 to deliver the electrical stimulation to the patient based on the received stimulation-evoked signals and/or composite stimulation-evoked signals, where the indications of the received stimulation-evoked signals and/or composite stimulation-evoked signals may be stored in a storage device. Processing circuitry 210A and/or 210B also controls stimulation generation circuitry 202 to generate and apply the stimulation signals to selected combinations of electrodes 232A, 232B.

In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232A, 232B. Such a switch circuit may selectively couple stimulation energy to selected electrodes 232A, 232B and to selectively sense bioelectrical neural signals of a sacral nerve or muscles of the patient with selected electrodes 232A, 232B. In other examples, however, stimulation generation circuitry 202 does not include a switch circuit and switch circuitry 204 does not interface between stimulation generation circuitry 202 and electrodes 232A, 232B. In these examples, stimulation generation circuitry 202 may include a plurality of pairs of current sources and current sinks, each connected to a respective electrode of electrodes 232A, 232B. In other words, in these examples, each of electrodes 232A, 232B is independently controlled via its own stimulation circuit (e.g., via a combination of a regulated current source and sink), as opposed to switching stimulation signals between different electrodes of electrodes 232A, 232B.

Storage device 212A and/or 212B may be configured to store information within IMD 200A and/or 200B, respectively, during operation. Storage device 212A and/or 212B may include a computer-readable storage medium or computer-readable storage device. In some examples, storage device 212A and/or 212B includes one or more of a short-term memory or a long-term memory. Storage device 212A and/or 212B may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212A and/or 212B is used to store data indicative of instructions, e.g., for execution by processing circuitry 210A and/or 210B, respectively. As discussed above, storage device 212A and/or 212B is configured to store stimulation parameter settings 242.

Power source 224 is configured to deliver operating power to the components of IMD 200A and/or 200B. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200A and/or 200B. Power source 224 may include any one or more of a plurality of different battery types, such as nickel cadmium batteries and lithium ion batteries.

In some examples as shown in FIG. 2, the processing circuitry 210A of the IMD 200A, and the processing circuitry 210B of the IMD 200B, controls and/or directs delivery of electrical stimulation by the electrodes 232A, 232B of leads 230A, 230B, receives stimulation-evoked signal data and/or information from sensors 222, and generates output based on the received data and/or information.

Processing circuitry 210A and/or 210B controls stimulation circuitry 202 to deliver stimulation energy with stimulation parameters specified by one or more stimulation parameter settings 242 stored on storage device 212A and/or 212B and, in the example of FIG. 2, to collect stimulation-evoked signals pertaining to the stored stimulation parameter settings 242. Processing circuitry 210A and/or 210B collects this stimulation-evoked signal information and/or composite stimulation-evoked signal information by receiving the information via sensing circuitry 206 and/or sensors 222.

Processing circuitry 210A may also control stimulation circuitry 202 to test different parameter settings and record one or more corresponding stimulation-evoked signals for each selected combination, and test different parameter settings as compared to one or more sensed stimulation-evoked signals.

In accordance with the devices and techniques disclosed herein, processing circuitry 210A and/or 210B may be configured to determine characteristics of two or more sensed stimulation-evoked signal types in one or more sensed stimulation-evoked signals that are evoked from delivery of electrical stimulation (e.g., by sweeping through stimulation signals meant to evoke the stimulation-evoked signals). Processing circuitry 210A and/or 210B may then utilize the characteristics to determine stimulation signal to evoke subsequent stimulation-evoked signals or to sense subsequent stimulation-evoked signals.

For example, processing circuitry 210A directs stimulation circuitry 202 to deliver stimulation via electrical stimulation settings, and the signal unit 216 collects the corresponding stimulation-evoked signal data 254 from sensing circuitry 206 via sensing settings. In some examples, processing circuitry 210A may direct stimulation circuitry 202 to deliver electrical stimulation via sweeping through a plurality of electrical stimulation settings, and processing circuitry 210A may direct sensing circuitry 206 to sense stimulation-evoked evoked signals evoked by the delivered electrical stimulation via sweeping through a plurality of sensing settings or parameters, e.g., an electrode configuration such as monopolar, bipolar, tripolar, or the like, alternating sensing polarity, blanking parameters, or any other suitable sensing parameter. In some examples, the electrical stimulation that processing circuitry 210A directs stimulation circuitry 202 to deliver may additionally provide therapeutic electrical stimulation.

Processing circuitry 210A may determine characteristics of one or more, or two or more, different signal types (e.g., stimulation-evoked signal types) that are received in response to using one or more settings selected from electrical stimulation settings, sensing settings, or combinations thereof, and program the electrical stimulation system (e.g., IMD 200A), based on the characteristics. For example, based on the determined characteristics, processing circuitry 210A may program IMD 200A to include at least one of electrical stimulation settings for delivering subsequent electrical stimulation to the patient for evoking at least one of the signal types, or sensing settings for sensing at least one of the signal types in response to stimulation of the patient. Processing circuitry 210A may then direct stimulation circuitry 202 to deliver, according to the determined electrical stimulation settings, the subsequent electrical stimulation to the patient to evoke the one or more subsequent stimulation-evoked signals from the patient. Additionally or alternatively, processing circuitry 210A may direct sensing circuitry 206 to sense, according to the determined sensing settings, the one or more subsequent stimulation-evoked signals from the patient in response to the subsequent electrical stimulation.

For example, processing circuitry 210A may be configured to determine an operating mode best suited for a particular patient. For example, processing circuitry 210A may capture the stimulation-evoked signals and subsequent stimulation-evoked signals via a calibration process, and determine an operating mode based on the determined stimulation-evoked signal types along with the electrical stimulation and sensing settings corresponding to generating the stimulation-evoked signals and subsequent stimulation-evoked signals. For example, processing circuitry 210A may determine an ECAP mode, an EMG mode, an alternating ECAP then EMG mode, a concurrent ECAP and EMG mode, or any other operating mode suitable to improve and/or optimize evoking and sensing stimulation-evoked signals which provide information indicative of the efficacy of delivering therapeutic electrical stimulation to the patient.

In some examples, processing circuitry 210A may deliver therapeutic electrical stimulation based on the one or more subsequent stimulation-evoked signals. For example, processing circuitry 210A may run a calibration, determine an operating mode, and deliver therapeutic electrical stimulation according to electrical stimulation and sensing settings optimized for the operating mode. In some examples, processing circuitry 210A may direct sensing circuitry 206 to sense stimulation-evoked signals from the delivery of therapeutic stimulation. In some examples, processing circuitry 210A may direct sensing circuitry 206 to not sense stimulation-evoked signals from therapeutic stimulation, e.g., to only sense stimulation-evoked signals from delivery of electrical stimulation according to a calibration or a parameter sweep. For example, processing circuitry 210A may determine electrical stimulation and sensing settings based only on stimulation-evoked signals evoked by electrical stimulation designed to evoke stimulation-evoked signals and/or particular stimulation-evoked signal types.

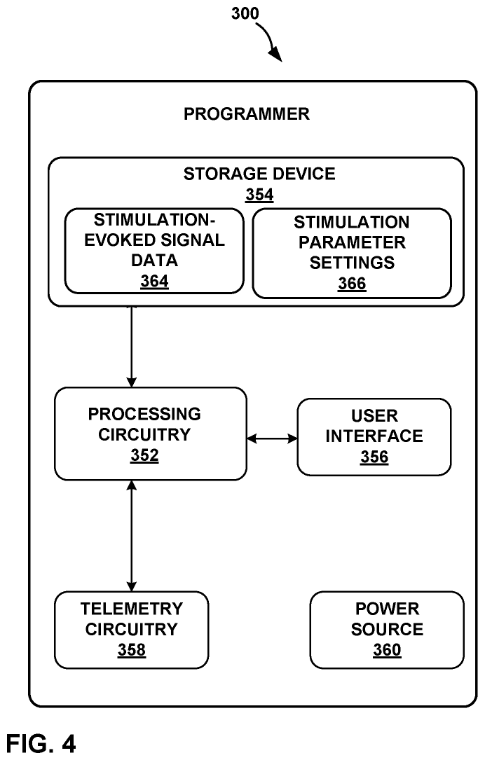

FIG. 4 is a block diagram illustrating an example configuration of components of an example external programmer 300. External programmer 300 may be an example of external device 24 of FIG. 1. Although external programmer 300 may generally be described as a hand-held device, such as a tablet computer or smartphone-like device, external programmer 300 may be a larger portable device, such as a laptop computer, or a more stationary device, such as a desktop computer. In addition, in other examples, external programmer 300 may be included as part of an external charging device or include the functionality of an external charging device, e.g., to recharge a battery or batteries associated with IMD 200. As illustrated in FIG. 4, external programmer 300 may include processing circuitry 352, storage device 354, user interface 356, telemetry circuitry 358, and power source 360. In some examples, storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include processing circuitry configured to perform the processes discussed with respect to processing circuitry 352.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, processing circuitry 352, telemetry circuitry 358, or other circuitry of external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352, telemetry circuitry 358 or other circuitry of external programmer 300 may correspond to individual hardware units, such as ASICs, DSPs, FPGAs, or other hardware units.

The processing circuitry 352 is configured to direct delivery of electrical stimulation, receive information relating to one or more stimulation-evoked signal(s). In some examples, the processing circuitry 352 is configured to control the electrical stimulation circuitry to deliver the electrical stimulation based on the received stimulation-evoked signal information in a closed loop basis by directing the IMD to use particular stimulation parameters.

In some examples, storage device 354 may include instructions that cause processing circuitry 352 to obtain a parameter set from memory or receive user input and send a corresponding command to IMD 200, or instructions for any other functionality. In addition, storage device 354 may include a plurality of programs, where each program includes a parameter set that defines therapy stimulation or control stimulation. Storage device 354 may also store data received from a medical device (e.g., IMD 16) and/or a remote sensing device. For example, storage device 354 may store data recorded at a sensing module of the medical device, and storage device 354 may also store data from one or more sensors of the medical device. In an example, storage device 354 may store data recorded at a remote sensing device such as one or more stimulation-evoked signal sensed by one or more sensors.

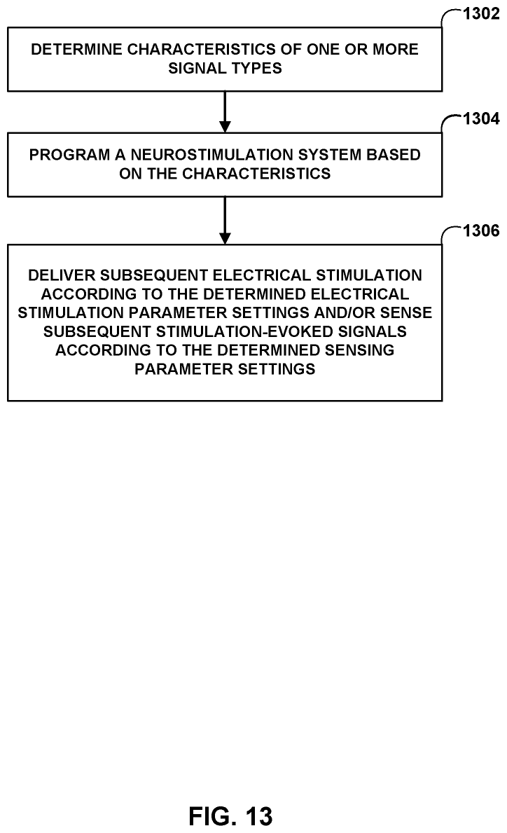

User interface 356 may include a button or keypad, lights, a speaker for voice commands, a display, such as a liquid crystal (LCD), light-emitting diode (LED), or organic light-emitting diode (OLED). In some examples, the display includes a touch screen. User interface 356 may be configured to display any information related to the delivery of electrical stimulation including output, for example, information based on one or more stimulation-evoked signal. User interface 356 may also receive user input (e.g., indication of when the patient perceives stimulation, or a pain score perceived by the patient upon delivery of stimulation) via user interface 356. The user input may be, for example, in the form of pressing a button on a keypad or selecting an icon from a touch screen. The input may request starting or stopping electrical stimulation, the input may request a new electrode combination or a change to an existing electrode combination, or the input may request some other change to the delivery of electrical stimulation, such as a change in electrode combination or configuration, electrode polarity, amplitude, pulse width, pulse shape, pulse frequency or pulse rate, cycling, or biphasic recharge parameters, or pulse patterns (e.g., masker-probe). In some examples, the input may cause, or trigger, a sensing or calibration sequence, e.g., as described below at FIGS. 13-15.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 16 include RF communication according to the Bluetooth ® or 802.11 specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameters to IMD 16 for delivery of electrical stimulation therapy.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

In some examples, the external programmer 300 directs delivery of electrical stimulation of an IMD, receives information relating to stimulation-evoked signals and/or composite stimulation-evoked signals, and generates output based on the received information, e.g., for evaluation of efficacy of stimulation parameters and/or to recommend or assist a user in programming stimulation parameters for delivery of electrical stimulation, or used as part of a closed loop control scheme to automatically adjust stimulation parameters using stimulation-evoked signal information and/or composite stimulation-evoked signal information. In one or more examples, the external programmer 300 generates output based on stimulation-evoked signal information, e.g., output which may be used as part of closed loop control, output which may be displayed and used by external programmer 300 to manually control therapy delivery, output which may be used to maintain delivery of the same therapy, output which may be recorded and tracked, or output which may be suitable for any other purpose relating to delivery of electrical stimulation therapy.

Programmer 300 may be a patient programmer or a clinician programmer and receives stimulation-evoked signal information and/or composite stimulation-evoked signal information such as stimulation-evoked signal data 364. Programmer 300 receives stimulation-evoked signal(s) information and allows a user to interact with the processing circuitry 352 via user interface 356 in order to identify parameter settings, such as cycling and/or one or more other stimulation parameters using the stimulation-evoked signal information. Programmer 300 further assists the user in programming a neurostimulation device by using the stimulation-evoked signal information displayed on the user interface 356. In addition, programmer 300 may be used as part of a closed loop control scheme to automatically adjust stimulation parameters based at least on stimulation-evoked signal information. In some examples, programmer 300 receives stimulation-evoked signal information and/or composite stimulation-evoked signal information such as stimulation-evoked signal data 364 from one or more sensor devices and stores the stimulation-evoked signal data 364 in the storage device 354. In some examples, programmer 300 may be device specifically made to communicate with an IMD, e.g., IMD 16, IMD 200A, IMD 200B, and the like, as part of an electrical stimulation system. In other examples, programmer 300 may be a device configured to interact with an IMD or other device of an electrical stimulation system, e.g., a computing device and/or mobile phone configured to run suitable application software for the electrical stimulation system and configured to communicate with one or more devices, e.g., an IMD, of the electrical stimulation system.

Programmer 300 may be used to determine efficacy of particular parameter settings of the IMD by testing parameter settings and recording one or more stimulation-evoked signal for each parameter setting. For example, programmer 300 may be used to cause the IMD to automatically scan though a plurality of electrode combinations or parameter combinations. Processing circuitry 352 causes the IMD to automatically scan through each of a plurality of parameter combinations, including electrode combinations and parameter combinations. For each combination, the programmer 300 obtains and records one or more corresponding stimulation-evoked signal and/or composite stimulation-evoked signal. In some examples, programmer 300 may be used to cause the IMD to automatically scan through a plurality of electrode combinations or parameter combinations at one or more times, e.g., periodically every hour, day, week, month, year, and/or non-periodically, e.g., according to a schedule or other determination of when to repeat a scan, and obtains and records one or more corresponding stimulation-evoked signal and/or composite stimulation-evoked signal for each scan. In some examples, programmer 300 or another device, e.g., IMD 16, external device 24, server 26, or other device, may compare the recorded stimulation-evoked signal and/or composite stimulation-evoked signal over time.

Alternative to or in addition to the automatic scanning process, the user could manually advance scanning through electrode pairs and/or parameter combinations, for example with an arrow button on user interface 356. In some examples, as the user scans through the electrode pairs or parameter combinations to test and record one or more stimulation-evoked signal for each combination.

Processing circuitry 352 controls stimulation circuitry 202 to deliver stimulation energy with stimulation parameters specified by one or more stimulation parameter settings 366 stored on storage device 354, and to collect stimulation-evoked signal information pertaining to the stored stimulation parameter settings 366. For example, processing circuitry 352 may be substantially similar to processing circuitry 210A and/or 210B described above, except located in an external programmer 300.

The architecture of external programmer 300 illustrated in FIG. 4 is shown as an example. The techniques as set forth in this disclosure may be implemented in the example external programmer 300 of FIG. 4, as well as other types of systems not described specifically herein. Nothing in this disclosure should be construed so as to limit the techniques of this disclosure to the example architecture illustrated by FIG. 4.

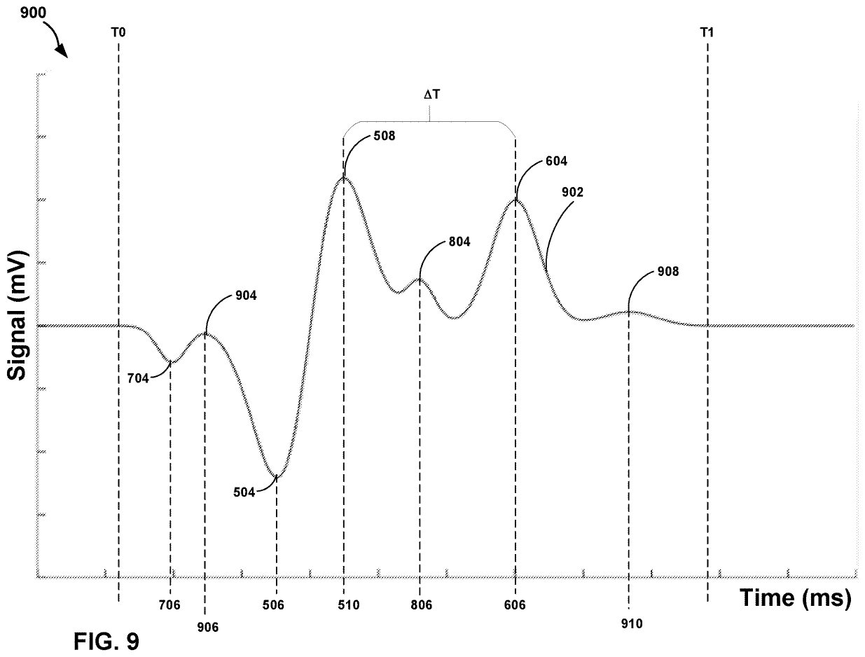

FIGS. 5-8 are plots of example stimulation-evoked signals and FIGS. 9-11 are example composite stimulation-evoked signals, and are described together below. In the specific examples of FIGS. 5-11 below, each signal plotted represent a voltage amplitude of a circuit including an electrode 232 that varies in time in proportion to a time-varying electric field sensed by the electrode 232. The time-varying field in the examples shown is caused by one or more signal sources, e.g., nerve, muscle, or other tissue, of a patient in response to electrical stimulation. However, FIGS. 5-11 may generally represent one or more other quantities. In some examples, each signal plot may represent an amplitude as a function of time of a sensed quantity over time, the quantity varying in proportion to a physiological response of a signal source. In some examples, the quantity is an amplitude measured by a sensor. For example, the amplitude may be a voltage and/or current that varies in time according to an amplitude of an electric field and/or potential emitted and/or induced by a signal source. In some examples, the amplitude may be a displacement, a pressure, accelerometer data, a sound, e.g., such as an MMG signal. In some examples, composite stimulation-evoked signal 902 described below may be a composite of sensed quantities from a plurality of sources sensed by a plurality of sensors, e.g., combined amplitudes as a function of time from two or more different sensors sensing two or more different quantities from one or more different signal sources that respond to the same electrical stimulation at or near the same time or within a period of time (e.g., a sensing “time window”). In some examples, two sensors may sense two different quantities from the same signal source, e.g., an EMG and an MMG of a muscle response. In other examples, composite stimulation-evoked signal 902 may be a composite of a sensed quantity, e.g., an electric field and/or potential, from a plurality of signal sources sensed by the same sensor, e.g., an electrode 232 sensing a varying electric field that is a superposition of a plurality of electric fields caused by a plurality of signal sources responding to electrical stimulation within a sensing time window.

FIG. 5 is a plot 500 of an example stimulation-evoked signal 502, in accordance with one or more techniques of this disclosure. In the example shown, signal 502 is a voltage amplitude that varies in time in proportion to a time-varying electric field sensed by an electrode 232, the time-varying electric field caused by a signal source in response to electrical stimulation. In the example shown, time TO corresponds to a time at which electrical stimulation of a nerve or muscle ceases, e.g., is turned off, and time T1 corresponds to the ending time of the sensing time window, e.g., the sensing time window is the difference between TO and T1. In some examples, signal 502 may have a signal length in time that is equal to the time window, e.g., the physiological response of the signal source emits a detectable quantity (e.g., electric field) that lasts for the length of the time window. In other examples, the signal length of signal 502 may be less than the time window. Generally, the time window may be chosen based on signal length, e.g., time TO may be chosen to be the time at which electrical stimulation ceases and time T1 may be chosen based on the time-length of the sensed signal, e.g., any of 502, 602, 702, 802, and/or 902. In the examples of FIGS. 5-9, T1 chosen based on an exemplary time-length of signal 902 and is shown on each of plots 500-900 for reference. In some examples, the length of stimulation-evoked signals 502-902 may be relatively long, e.g., 1 ms, 5 ms, 10 ms, 15 ms, 20 ms, 30 ms, or longer. In some examples, the shape, length, and location along the time axis of one or more features of stimulation-evoked signals 502-802 may be different.

In the example shown, signal 502 includes valley 504 (which may be considered a “peak” with a negative amplitude and may be simply referred to as a “peak” herein) at time 506 and peak 508 at time 510. In the example shown, signal 502 may be a stimulation-evoked signal of an EMG response of a muscle to electrical stimulation.

FIG. 6 is a plot 600 of another example stimulation-evoked signal 602, in accordance with one or more techniques of this disclosure. In the example shown, signal 602 is a voltage amplitude that varies in time in proportion to a time-varying electric field sensed by an electrode 232, the time-varying electric field caused by a signal source in response to electrical stimulation. In the example shown, signal 602 includes peak 604 at time 606. In the example shown, signal 602 may be a stimulation-evoked signal of an EMG of a muscle in response to electrical stimulation.

FIG. 7 is a plot 700 of another example stimulation-evoked signal 702, in accordance with one or more techniques of this disclosure. In the example shown, signal 702 is a voltage amplitude that varies in time in proportion to a time-varying electric field sensed by an electrode 232, the time-varying electric field caused by a signal source in response to electrical stimulation. In the example shown, signal 702 includes valley 704 at time 706. In the example shown, signal 702 may be a stimulation-evoked signal of a neural response of nerve fibers to electrical stimulation.

FIG. 8 is a plot 800 of another example stimulation-evoked signal 802, in accordance with one or more techniques of this disclosure. In the example shown, signal 802 is a voltage amplitude that varies in time in proportion to a time-varying electric field sensed by an electrode 232, the time-varying electric field caused by a signal source in response to electrical stimulation. In the example shown, signal 802 includes peak 804 at time 806. In the example shown, signal 802 may be a stimulation-evoked signal of a neural response of one or more fibers of a nerve or an EMG of a muscle in response to electrical stimulation.

FIG. 9 is a plot of an example composite stimulation-evoked signal, in accordance with one or more techniques of this disclosure. In the example shown, signal 902 is a voltage amplitude that varies in time in proportion to a time-varying electric field sensed by an electrode 232, the time-varying electric field caused by a plurality of signal sources in response to electrical stimulation. For example, signal 902 may be a composite of signals 502-802. Although not shown, signal 902 may include other peaks, features, artifacts, and/or noise. For example, electrode 232 may sense signal 902 but not signals 502-802, which are illustrated for as individual components of composite signal 902 for clarity.

In the example shown, composite stimulation-evoked signal 902 includes peaks 504, 508, 604, 704, 804, and 904 and 908 occurring at times 506, 510, 606, 706, 806, and 906 and 910, respectively. In the example shown, peak 904 may correspond to a combination of two or more signal sources. In other words, peak 904 may not be a peak caused by a signal source, but rather is a result of the combination of signals 502 and 702. Peak 908 may be a stimulation-evoked signal of an EMG of a muscle in response to electrical stimulation, e.g., a second contraction of the same muscle of peak 604 or a different muscle.

In some examples, a plurality of features of signal 902 may be determined, e.g., per (406) of the method illustrated and described above with reference to FIG. 4B. For example, IMD 200A, external programmer 300, or another device such as a computing device, may determine receive signal 902 and determine one or peaks 504, 508, 604, 704, 804, 904 and 908, the corresponding times of the peaks, latency between one or more peaks such as AT between peak 508 and 604, the widths and areas of any of the above peaks, the frequency and/or spectral content of signal 902, or any other signal feature, e.g., derivable via signal processing and/or digital signal processing.

In some examples, one or more determined feature may correspond to, and may be correlated with, the efficacy of stimulation therapy. For example, peak 504 may relate to an electrical stimulation response of certain fibers of a nerve to electrical stimulation, peak 604 may relate to an EMG of a muscle, and peak 704 may relate to an electrical stimulation response of nerve fibers, e.g., which may relate to sensory and motor information. In some examples, improved and or optimal electrical stimulation therapy may be electrical stimulation that excites certain nerve fibers while reducing/minimizing excitation of certain other nerve fibers, e.g., such that peak 508 is increased and peak 704 is decreased. For example, a system may determine that leads 230 may be moved and/or stimulation parameters settings 242 may be adjusted to increase peak 508 (e.g., increase excitation of the certain nerve fibers) while also decreasing peak 704 (e.g., reducing valley 704 or making peak 704 less negative, representing a decrease of the excitation of certain other nerve fibers).

As another example, improved and or optimal electrical stimulation therapy may be electrical stimulation that reduces/minimizes fiber excitation of some fibers while increasing excitation of other nerve fibers and muscle contraction, e.g., the EMG response of a muscle. For example, a system may determine that leads 230 may be moved and/or stimulation parameters settings 242 may be adjusted to increase peak 704 (e.g., increase valley 704 or make peak 704 more negative, representing an increase of the excitation of certain fibers of a nerve) while increasing peak 604 (e.g., increasing the response and corresponding EMG of a muscle) and decreasing peak 508 (e.g., decreasing excitation of other fibers of a nerve).

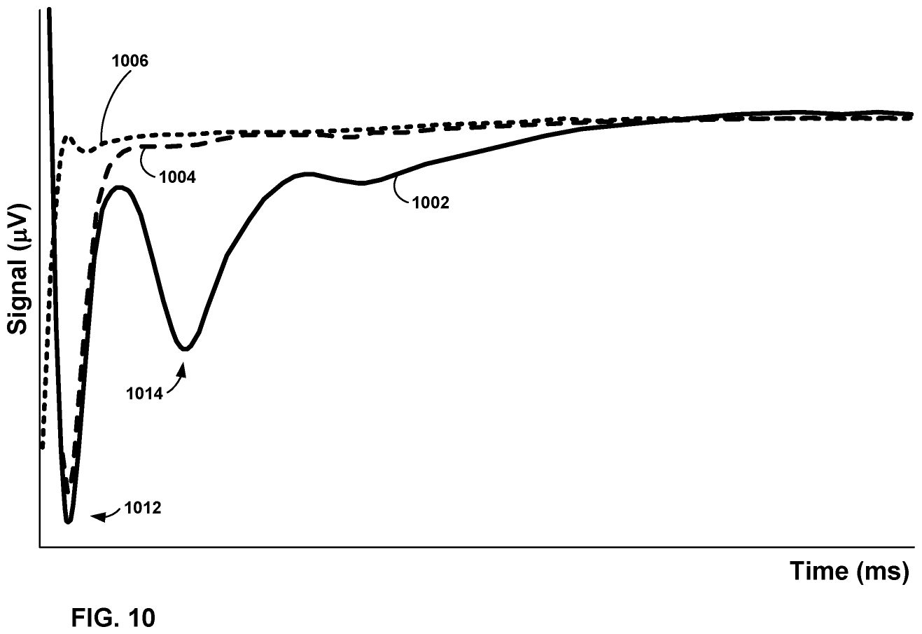

FIG. 10 is a plot of other example stimulation-evoked signals, in accordance with one or more techniques of this disclosure. In the example shown, signals 1002, 1004, and 1006 are voltage amplitudes that vary in time in proportion to a time-varying electric field sensed by an electrode 232, the time-varying electric field caused by a one or more signal sources in response to electrical stimulation. In the example shown, signals 1002-1006 are stimulation-evoked signals sensed from sacral neuromodulation leads. Signal 1002 is a baseline composite stimulation-evoked signal, signal 1004 is a stimulation-evoked signal without a muscle response, and signal 1006 is a stimulation-evoked signal without a neural or muscle response. In the example shown, peaks 1012 and 1014 (e.g., which may be “negative peaks” or valleys) comprises features in composite signal 1002 from multiple sources, e.g., peak 1012 is a signal feature indicating a neural response, e.g., an ECAP, and peak 1014 is a signal feature indicating a muscle response, e.g., an EMG or a compound muscle action potential (CMAP). In the example shown, signal 1004 includes peak 1012, e.g., an ECAP signal, but does not include peak 1014. Signal 1006 does not include ECAP or EMG signal features.

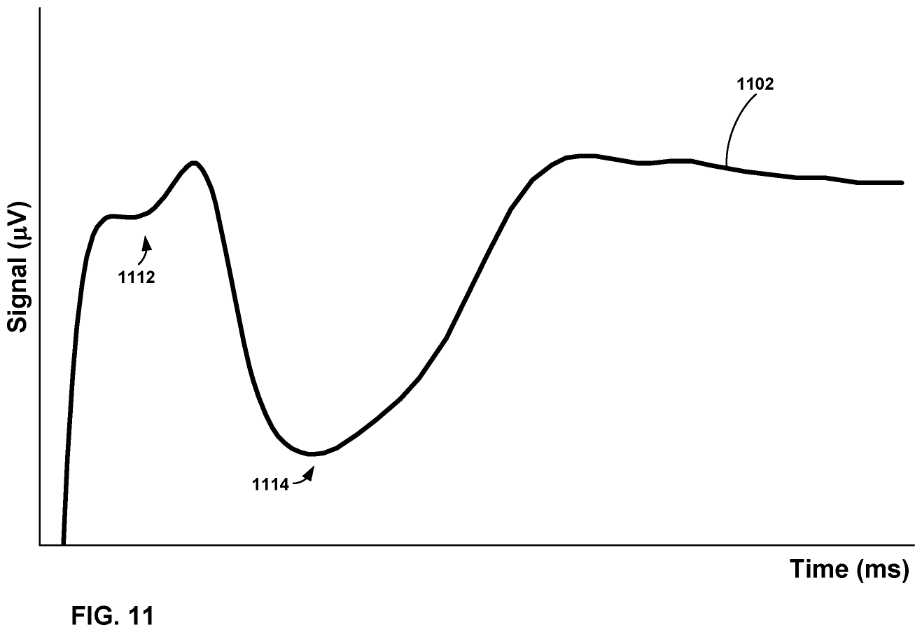

FIG. 11 is a plot of another example composite stimulation-evoked signal, in accordance with one or more techniques of this disclosure. In the example shown, signal 1102 is a voltage amplitude that varies in time in proportion to a time-varying electric field sensed by an electrode 232, the time-varying electric field caused by a one or more signal sources in response to electrical stimulation. In the example shown, signal 1102 is a composite stimulation-evoked signal sensed from sacral neuromodulation. In the example shown, peaks 1112 and 1114 comprises features in composite signal 1102 from multiple sources, e.g., peak 1112 is a signal feature indicating a neural response, e.g., an ECAP, and peak 1114 is a signal feature indicating a muscle response, e.g., an EMG or a CMAP.

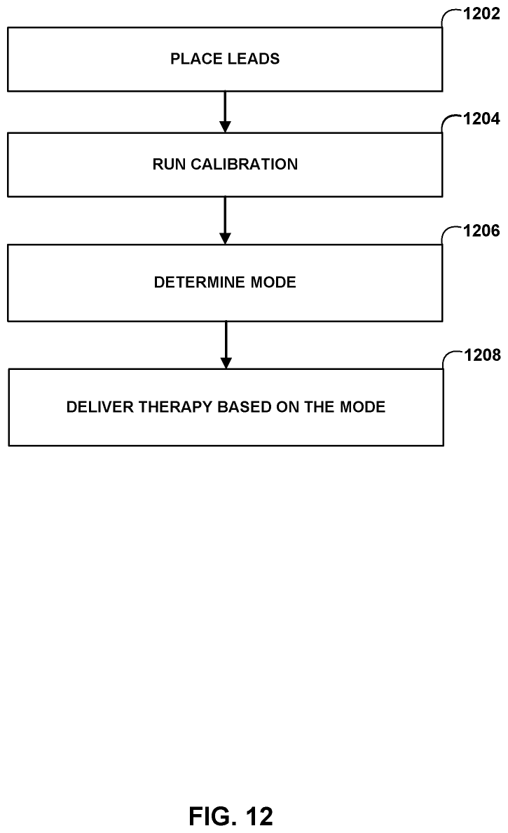

FIG. 12 is a flow diagram illustrating an example method of controlling delivery of electrical stimulation therapy, in accordance with one or more techniques of this disclosure. Although FIG. 12 is discussed using IMD 200A of FIG. 2, and external programmer 300 of FIG. 4, it is to be understood that the methods discussed herein may include and/or utilize other systems and methods in other examples. In some examples, IMD200A may perform the method of FIG. 12 to determine characteristics of two or more sensed stimulation-evoked signal types in one or more sensed stimulation-evoked signals that are evoked from delivery of electrical stimulation (e.g., by sweeping through stimulation signals meant to evoke the stimulation-evoked signals), and then utilize the characteristics to determine stimulation signal to evoke subsequent stimulation-evoked signals or to sense subsequent stimulation-evoked signals.

A physician or clinician may place electrical stimulation leads (12020). For example, a physician or clinician may implant one or more leads 18, 20, or 28 within patient 14 to deliver electrical stimulation to a target tissue.

IMD 200A may run a calibration sequence (1204). For example, IMD 200A may run a calibration to tailor an operating mode for a particular patient, e.g., an ECAP mode, an EMG mode, an alternating ECAP then EMG mode, a concurrent ECAP and EMG mode, or the like. For example, IMD 200A may determine a calibration is needed, or a physician, clinician, or patient may request a calibration. IMD200A may run a calibration sequence by delivering electrical stimulation (e.g., via electrodes of leads 18, 20, or 28) according to a plurality of sets of electrical stimulation settings, e.g., varying electrode configurations and polarities, varying amplitudes, varying frequencies, and/or varying frequencies. IMD 200A may sense and record (e.g., captured, detect, process, and/or store) stimulation-evoked signals in response to the delivered electrical stimulation according to a plurality of sets of sensing settings.

IMD 200A may determine an operational mode of a therapy system, e.g., therapy system 10 (1206). For example, based on one or more stimulation-evoked signals recorded for known electrical stimulation settings and sensing settings, IMD 200A may determine one or more characteristics of stimulation-evoked signal types, e.g., ECAP signals, EMG signals, or any suitable stimulation-evoked signal types. In some examples, IMD 200A may determine one or more features of one or more recorded stimulation-evoked signals, and determine a signal type of one or more of the stimulation-evoked signals and/or one or more characteristics of one or more of the stimulation-evoked signals and signal types.

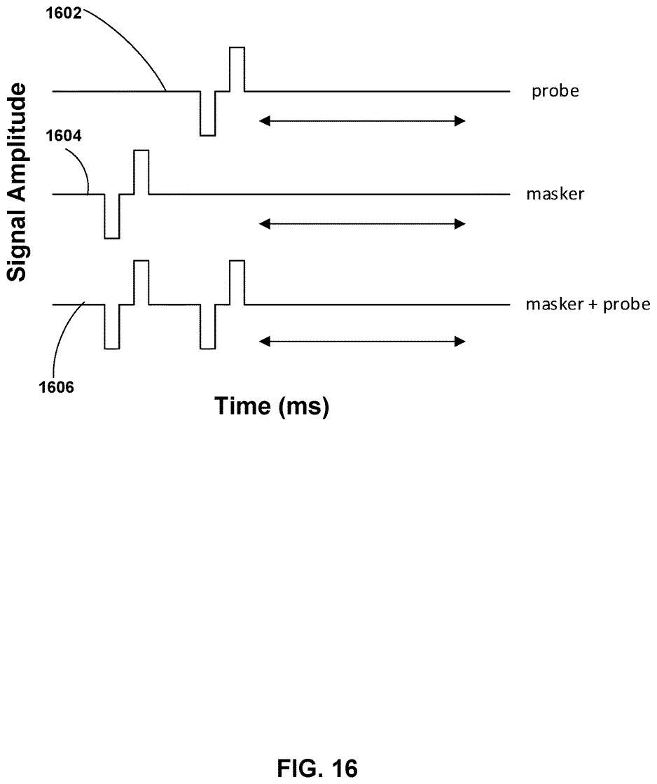

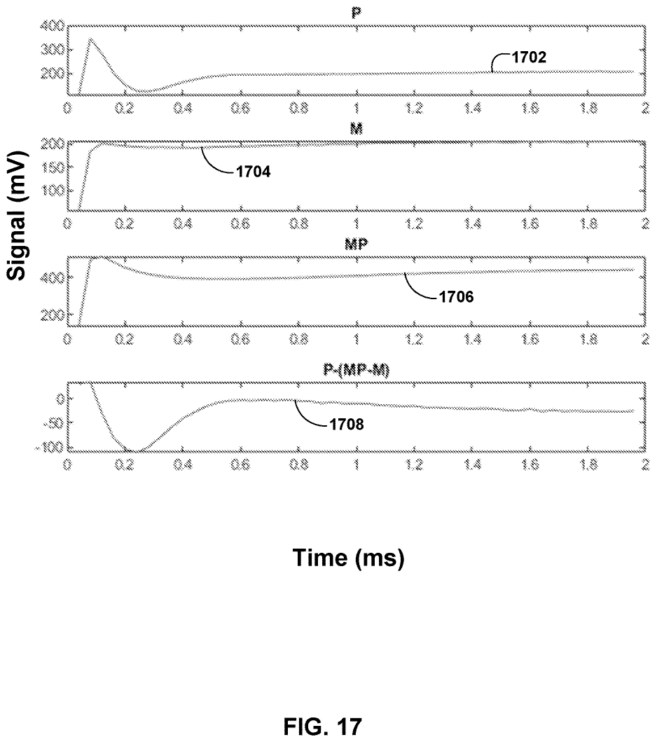

IMD 200A may then determine an operational mode based on the determined characteristics. For example, IMD 200A may determine an ECAP operational mode in which electrical stimulation settings and sensing settings are selected to optimize evoking and sensing of ECAP signals, e.g., as compared to EMG signals. Alternatively, IMD 200A may determine an EMG operational mode in which electrical stimulation settings and sensing settings are selected to optimize evoking and sensing of EMG signals, e.g., as compared to ECAP signals. In some examples, IMD 200A may determine an operational mode including stimulation/sensing schemes that are compatible with stimulation-evoked signals of a particular type. For example, if IMD 200A determines an ECAP operational mode, the ECAP operational mode may include a masker-probe technique (FIGS. 16 and 17) configured to reduce stimulation artifacts from the recording of ECAPs. If IMD 200A determines an EMG operational mode, the EMG operational mode may include an alternating polarity technique configured to change pulse polarity on every other pulse and averaging the pulses. Alternatively still, IMD 200A may determine an alternating operational mode in which electrical stimulation settings and sensing settings are selected to optimize evoking and sensing a first stimulation-evoked signal type (e.g., ECAPs) followed by electrical stimulation settings and sensing settings selected to optimize evoking and sensing a second stimulation-evoked signal type (e.g., EMG signals). Also, IMD 200A may determine a concurrent operational mode in which electrical stimulation settings and sensing settings are selected to optimize evoking and sensing a plurality of stimulation-evoked signal types, e.g., a composite stimulation-evoked signal comprising ECAP and EMG signals.

IMD 200A may deliver therapy based on the determined mode (1208). For example, IMD 200A may determine electrical stimulation settings and sensing settings based on the determined mode, and deliver subsequent electrical stimulation, and sense subsequent stimulation-evoked signals, according to the determined electrical stimulation settings and sensing settings, e.g., to deliver one-time or chronic electrical stimulation therapy.

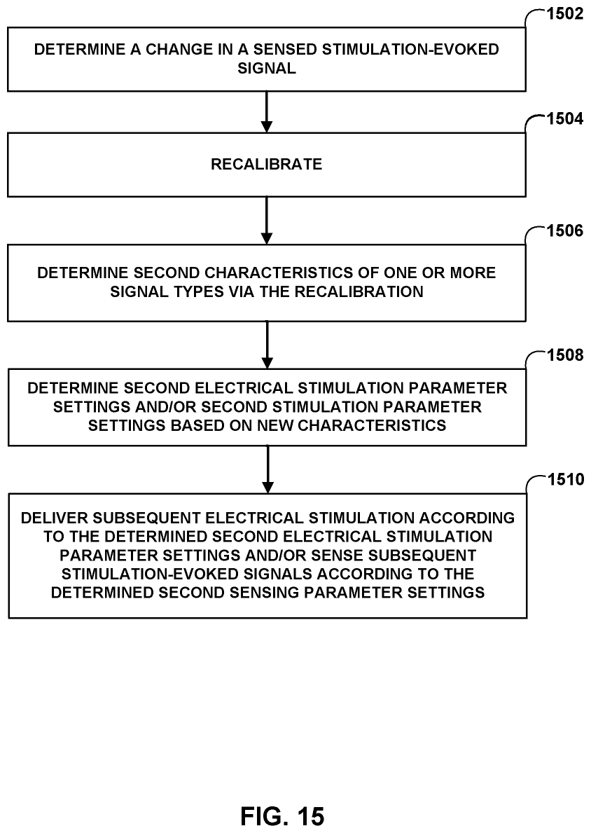

In some examples, IMD 200A may operate in closed loop within a selected operational mode. For example, IMD 200A may determine an operational mode giving improved and/or optimized stimulation-evoked signals from which to determine the efficacy of the electrical stimulation therapy. IMD 200A may change electrical stimulation settings and/or sensing settings to change or improve the efficacy of the electrical stimulation therapy based on the stimulation-evoked signals of the type or types of the selected mode, e.g., based on characteristics and/or features of the sensed stimulation-evoked signals of the type or types of the selected mode. For example, within an operational mode, IMD 200A may update electrical stimulation settings and/or sensing settings to change or improve the efficacy of the electrical stimulation over time. In some examples, IMD 200A may recalibrate, or update a calibration, to determine if the same or a different operational mode would be advantageous to select at a future time, e.g., if the stimulation-evoked signals drift or change over time, as further described below with reference to FIG. 15.

FIG. 13 is a flow diagram illustrating an example method of controlling delivery of electrical stimulation therapy, in accordance with one or more techniques of this disclosure. Although FIG. 13 is discussed using IMD 200A of FIG. 2, and external programmer 300 of FIG. 4, it is to be understood that the methods discussed herein may include and/or utilize other systems and methods in other examples.

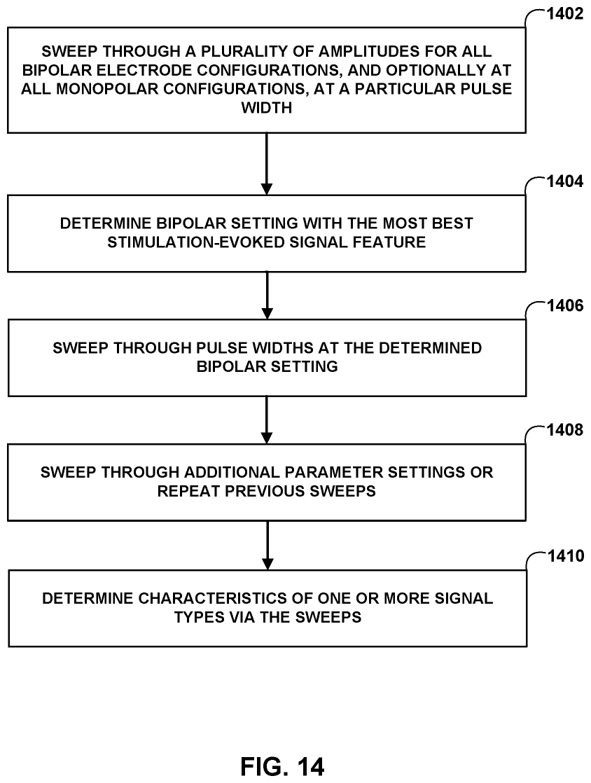

IMD 200A may determine, for a patient, characteristics of two or more sensed stimulation-evoked signal types that are received in response to using at least two settings selected from electrical stimulation settings, sensing settings, or combinations thereof (1302). For example, IMD 200A may run a calibration sequence as described above at (1204), or as further described below with reference to one or both of FIGS. 14 and 15.

For example, IMD200A may deliver electrical stimulation (e.g., via electrodes of leads 18, 20, or 28) by sweeping through (or causing electrodes of leads 18, 20, or 28 to sweep through) a delivery of a plurality of electrical stimulation pulses, where each electrical stimulation pulse is characterized by at least an electrode configuration, a pulse width, a polarity, a frequency, an amplitude a recharge parameter (e.g., a duration and/or amplitude of an active recharge or a duration and/or amplitude of a passive recharge), or a pulse pattern (e.g., such as a masker-probe sequence). For example, IMD 200A may deliver electrical stimulation by sweeping through a plurality of pulses comprising at least one of a 20 microsecond pulse, a 40 microsecond pulse, a 60 microsecond pulse, an 80 microsecond pulse, a 100 microsecond pulse, a 180 microsecond pulse, a 190 microsecond pulse, a 200 microsecond pulse, a 210 microsecond pulse, a 220 microsecond pulse, a 300 microsecond pulse, or a 450 microsecond pulse, at one more monopolar electrode configuration and/or bipolar electrode configuration. In some examples, IMD 200A may deliver electrical stimulation by sweeping through a plurality of pulses comprising a pulse that is at least 20 microseconds and less than or equal to 450 microseconds. In some examples, IMD 200A may deliver the electrical pulses at alternating polarities.

IMD 200A may sense and record (e.g., captured, detect, process, and/or store) a stimulation-evoked signal after a corresponding electrical stimulation pulse of the plurality of electrical stimulation pulses. For example, IMD 200A may sense and record a set of a plurality of stimulation-evoked signals, each corresponding to a delivered pulse of the sweep of pulses. IMD 200A may characteristics of one or more of the set of stimulation-evoked signals, each characteristic comprising one or more signal features. In some examples, IMD 200A may sense and record stimulation-evoked signals by sweeping through sensing parameters to sense the stimulation-evoked signals, e.g., sweeping through an electrode configuration (monopolar, bipolar, tripolar, or the like), alternating sensing polarity, or sweeping through a blanking parameter.

In some examples, IMD 200A may determine signal features comprising at least one of a signal peak (e.g., peaks 508, 604, 804, 908, 1012, 1014, 1112, or 1114), a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley (e.g., valleys 504, 704, 1012, 1014, 1112, or 1114), a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve feature, a signal maximum rate of change feature, or a signal minimum rate of change feature.

IMD 200A may determine, based on the features, characteristics of an ECAP stimulation-evoked signal type (e.g., based on features indicating at least one of the sources of the stimulation-evoked signal is an ECAP source) and a non-EMG stimulation-evoked signal type (e.g., based on features indicating that an EMG source is likely not a source of the stimulation-evoked signal), or IMD 200A may determine, based on the features, characteristics of an EMG stimulation-evoked signal type (e.g., based on features indicating at least one of the sources of the stimulation-evoked signal is an EMG source) and a non-ECAP stimulation-evoked signal type (e.g., based on features indicating that an ECAP source is likely not a source of the stimulation-evoked signal), or IMD 200A may determine, based on the features, characteristics of an ECAP stimulation-evoked signal type and an EMG stimulation-evoked signal type, (e.g., based on features indicating at least one of the sources of the stimulation-evoked signal is an ECAP source and at least one of the sources of the stimulation-evoked signals is an EMG source). In other words, IMD200A may determine, based on the features, characteristic of a composite stimulation-evoked signal, e.g., in which an ECAP and an EMG signal are concurrent (and at least partially overlapping in time) or in which an ECAP and an EMG signal are consecutive (substantially non-overlapping in time).

IMD 200A may program and/or set its program, based on the characteristics, to include at least one of: electrical stimulation settings for delivering subsequent electrical stimulation to a patient for evoking at least one of the signal types, or sensing settings for sensing at least one of the signal types in response to stimulation of the patient (1304). In some examples, IMD 200A may determine electrical stimulation settings for delivering subsequent electrical stimulation configured to evoke stimulation-evoked signals for one or more stimulation-evoked signal types. For example, IMD 200A may determine electrical stimulation settings for delivering subsequent electrical stimulation configured to evoke an ECAP signal, evoke an EMG signal, evoke a composite ECAP and EMG signal, or evoke an ECAP signal followed by an EMG signal (or evoke an EMG signal followed by an ECAP signal). In some examples, the subsequent electrical stimulation may comprise electrical stimulation according to one or more modes, e.g., an ECAP mode, an EMG mode, a composite ECAP and EMG mode, or an alternating ECAP then EMG mode. In some examples, the subsequent electrical stimulation may comprise a portion of a calibration, or may comprise a portion of therapeutic electrical stimulation, e.g., after calibration and during normal, chronic electrical stimulation therapy delivery.

In some examples, IMD 200A may determine electrical stimulation settings and/or sensing settings based on one or more thresholds. For example, IMD 200A may determine electrical stimulation settings and/or sensing settings, based on the determined characteristics, that evoke one or more stimulation-evoked signal types (e.g., ECAP, EMG, composite) with a threshold minimum signal strength, a minimum signal-to-noise ratio, a minimum efficacy, or the like, but also with a threshold maximum electrical stimulation strength, amplitude, energy, or the like. For example, IMD 200A may determine electrical stimulation settings and/or sensing settings, based on the determined characteristics, that balance the efficacy and/or quality of sensing a stimulation-evoked signal of a stimulation-evoked signal type (ECAP, EMG, composite) for the amount of electrical stimulation delivered to evoke that stimulation-evoked signal and to provide efficacious electrical stimulation to the patient.

IMD 200A may deliver, according to the determined electrical stimulation settings, the subsequent electrical stimulation to the patient to evoke the one or more subsequent stimulation-evoked signals from the patient, and/or sense, according to the determined sensing settings, the one or more subsequent stimulation-evoked signals from the patient in response to the subsequent electrical stimulation (1306). For example, IMD 200A may deliver subsequent electrical stimulation and sense subsequent stimulation-evoked signals to further calibrate, or refine the calibration, or to deliver therapeutic electrical stimulation. In some examples, IMD 200A may then deliver therapeutic electrical stimulation based on the one or more subsequent stimulation-evoked signals, e.g., after refining, confirming, or the like, the calibration and/or electrical stimulation settings and/or sensing settings.

FIG. 14 is a flow diagram illustrating an example method of controlling delivery of electrical stimulation therapy, in accordance with one or more techniques of this disclosure. Although FIG. 14 is discussed using IMD 200A of FIG. 2, and external programmer 300 of FIG. 4, it is to be understood that the methods discussed herein may include and/or utilize other systems and methods in other examples. In some examples, the example method of FIG. 14 may be an example of determining characteristics of stimulation-evoked signals and/or signal types, e.g., method step 1302 of FIG. 13.

IMD 200A may sweep (or cause electrodes of leads 18, 20, and/or 28 to sweep) through a plurality of amplitudes for all bipolar electrode configurations of leads 18, 20, and/or 28 at alternating polarities and at a particular pulse width (1402). For example, IMD 200A may deliver, or sweep through, a plurality of electrical pulses by administering 20 pulses of 210 microsecond pulse width and alternating polarity (e.g., 40 pulses total), stating with a zero or near-zero amplitude, and increasing the amplitude for each alternating polarity pair to a maximum signal threshold that IMD 200A may output, or to a maximum amplitude that is less than the maximum capability of IMD 200A, e.g., chosen by a clinician, physician, and/or IMD 200A based on previous patient response or other input information. IMD 200A may sense, or capture, a stimulation-evoked signal and/or stimulation-evoked signals corresponding to one or more of the pulses. For example, IMD 200A may sense or capture an ECAP, an EMG, or a composite signal comprising an ECAP and an EMG consecutively or concurrently, after each individual pulse (e.g., the stimulation-evoked signals being evoked by the pulse) or after two or more pulses (e.g., the stimulation-evoked signals being evoked by two or more of the pulses). In some examples, IMD200A may repeat the sweep at method step 1402 at a plurality of frequencies.

In some examples, IMD 200A may optionally sweep (or cause electrodes of leads 18, 20, and/or 28 to sweep) through a plurality of amplitudes for all monopolar electrode configurations of leads 18, 20, and/or 28 at alternating polarities and at the particular pulse width, e.g., in addition to, or alternatively to, the bipolar sweep IMD 200A may determine the electrode configuration with the best (e.g., “highest quality”) stimulation-evoked signal and/or best (e.g., highest quality) stimulation-evoked signal feature or features (1404). In some examples, the best (e.g., “highest quality”) stimulation-evoked signal is the signal with the largest amplitude, e.g., the largest signal, and the best stimulation-evoked signal feature or features may be based on predetermined criteria (e.g., highest amplitude, largest or least area under a peak, smallest or largest latency, shortest or longest time between peaks, particular spectral content, or the like), thresholds, comparisons, or the like. In some examples, the best stimulation-evoked signal is the “highest quality” signal, e.g., the signal having the largest signal-to-noise ratio, the greatest number of signal features, the signal having the clearest and/or most distinguishable signal features, or any suitable signal quality metric indicative of the “best” or “highest quality” signal. In some examples, IMD 200A may determine a monopolar electrode configuration, a bipolar electrode configuration, a tripolar electrode configuration, or any other electrode configuration, as corresponding to the best stimulation-evoked signal and/or signal feature(s) at method step 1406 IMD 200A may sweep (or cause electrodes of leads 18, 20, and/or 28 to sweep) through a plurality of pulse widths at the determined electrode configuration (1406). For example, IMD 200A may sweep through 20 pulses for each pulse width for a 20 microsecond pulse width, a 40 microsecond pulse width, a 60 microsecond pulse width, an 80 microsecond pulse width, a 100 microsecond pulse width, a 200 microsecond pulse width, and a 450 microsecond pulse width, with the determined electrode configuration (e.g., bipolar, monopolar, tripolar, or other) and amplitude, and in some examples, the determined frequency from method step 1402.

IMD 200A may sweep (or cause electrodes of leads 18, 20, and/or 28 to sweep) through one or more additional electrical stimulation settings and/or sensing settings, or repeat any previous sweeps (1408). For example, IMD 200A may repeat any of the sweeps of method steps 1402, 1404, or 1406 if the sensed stimulation-evoked signals are weak or have low stability, or low signal-to-noise ratio, or do not contain signal features, or if IMD 200A determines to sense additional stimulation-evoked signals to increase a confidence in determining a mode or electrical stimulation settings or sensing parameter sensing settings, e.g., to deliver therapeutic electrical stimulation.

In some examples, IMD 200A may store sensed stimulation-evoked signals in a database, e.g., as a “look up table.” In some examples, IMD 200A may store stimulation-evoked signals and/or quantities derived from sensed stimulation-evoked signals, e.g., features and/or characteristics, which may be associated with and/or correlated to the patient and/or stimulation parameter settings and/or sensing settings.

IMD 200A may determine, for a patient, characteristics of one or more sensed stimulation-evoked signal types in one or more sensed stimulation-evoked signals that are evoked from delivery of electrical stimulation via the sweeps (1410). For example, IMD 200A may determine the characteristics as described above at method step 1302, where the one or more sensed stimulation-evoked signals are sensed, recorded, detected, and/or captured via the sweeps described above. In some examples, IMD 200A may determine, for a patient, characteristics of two or more sensed stimulation-evoked signal types in one or more sensed stimulation-evoked signals that are evoked from delivery of electrical stimulation via the sweeps. For example, IMD 200A may determine an ECAP signal type and a non-EMG signal type in a sensed stimulation-evoked signal, or an EMG signal type and a non-ECAP signal type, or a composite signal type, e.g., an EMG signal type and an ECAP signal type.

FIG. 15 is a flow diagram illustrating an example method of controlling delivery of electrical stimulation therapy, in accordance with one or more techniques of this disclosure. Although FIG. 15 is discussed using IMD 200A of FIG. 2, and external programmer 300 of FIG. 4, it is to be understood that the methods discussed herein may include and/or utilize other systems and methods in other examples. In some examples, the example method of FIG. 15 may be an example of recalibrating, e.g., based on a change such as lead migration, changes in patient responses, environmental changes, disease progression, or any change causing a change in stimulation-evoked signals sensed subsequent to a previous calibration and determination of an operational mode.

IMD 200A may determine a change in a sensed stimulation-evoked signal, e.g., such as the quality of the signal (1502). In some examples, IMD 200A may determine a change in a sensed stimulation-evoked signal indicative of lead migration, e.g., a decrease in signal amplitude, a change in a feature or feature shape, or the like.

IMD 200A may recalibrate (1504). For example, IMD 200A may perform a calibration or a modified calibration (e.g., fewer sweeps across a smaller range of parameter settings) according to the methods of any or all of FIGS. 12-14.

IMD 200A may determine, for a patient, characteristics of one or more sensed stimulation-evoked signal types in one or more sensed stimulation-evoked signals that are evoked from delivery of electrical stimulation via the recalibration (1506). In some examples, IMD 200A may determine, for a patient, characteristics of two or more sensed stimulation-evoked signal types in one or more sensed stimulation-evoked signals that are evoked from delivery of electrical stimulation via the recalibration. In some examples, IMD 200A may determine the characteristics as described above at method step 1302 and/or method step 1412, where the one or more sensed stimulation-evoked signals are sensed, recorded, detected, and/or captured via the recalibration sweeps of method step 1502.

For example, IMD 200A may determine, for the patient, second characteristics (at a later time from previously determined characteristics) of two or more sensed stimulation-evoked signal types in one or more sensed stimulation-evoked signals that are evoked from delivery of electrical stimulation of the recalibration or recalibration sweeps.

IMD 200A may determine, based on the characteristics, at least one of: second electrical stimulation settings for delivering subsequent electrical stimulation to a patient for evoking one or more subsequent stimulation-evoked signals, or second sensing settings for sensing one or more subsequent stimulation-evoked signals from the patient (1508). In some examples, IMD 200A may determine second electrical stimulation and/or sensing settings as described above at method step 1304, where the characteristics are the second characteristics determined via method stop 1506.

In some examples, IMD 200A may determine one or more different stimulation and/or sensing settings that compensate for a change in sensed stimulation-evoked signals, or that improve the efficacy of sensing stimulation-evoked signals or determining stimulation-evoked signal types (e.g., ECAP, non-ECAP, EMG, non-EMG, composite).

For example, IMD 200A may determine a different electrode combination for delivering electrical stimulation that compensates for drift and/or lead migration, e.g., to improve or optimize delivering electrical stimulation to a different location or sensing stimulation-evoked signals from a different location.

IMD 200A may deliver, according to the determined second electrical stimulation settings, the subsequent electrical stimulation to the patient to evoke the one or more subsequent stimulation-evoked signals from the patient, and/or sense, according to the determined second sensing settings, the one or more subsequent stimulation-evoked signals from the patient in response to the subsequent electrical stimulation (1510). In some examples, IMD 200A may deliver electrical stimulation and sense stimulation-evoked signals according to the second electrical and/or sensing settings as described above at method step 1306, where the second electrical and/or sensing settings are determined via method stop 1508.

FIG. 16 is a series of plots of example electrical stimulation pulses for executing a masker-probe technique, and FIG. 17 is a series of plots of example sensed stimulation-evoked signals resulting from the pulses of FIG. 16. In the example shown, each of sensed stimulation-evoked signals 1702-1706 correspond to each delivered pulse 1602-1606, e.g., pulse 1602 evokes signal 1702, pulse 1604 evokes signal 1704, pulse 1606 evokes signal 1706. Signal 1708 is mathematically computed from signals 1702, 1704 and 1706.

In some examples, a masker-probe technique may be used to reduce a stimulation artifact or artifacts from the recording of ECAPs. For example, IMD 200A may deliver (or cause an electrode of leads 18, 20, and/or 28 to delivery) three pulse sequences: probe only pulse 1602, masker pulse 1604, and masker and probe pulse 1606. IMD 200A may then sense and record the stimulation-evoked signal from each pulse, e.g., 1702, 1704, and 1706, and process the recorded signals, e.g., probe−(masker+probe)+masker to get the resultant ECAP with the artifact(s) removed, e.g. 1708. With the masker+probe sequence, when the pulses are close together, the neurons being activated may be in a relative refractory period after the probe pulse such that a small (or no) ECAP is elicited, while the artifact is still present, allowing the artifact to be subtracted from the recorded probe only ECAP.

The following numbered examples may illustrate one or more aspects of this disclosure:

Example 1: A method including: determining, for a patient, characteristics of one or more different signal types that are received in response to using at least one setting selected from the group of one or more electrical stimulation settings, one or more sensing settings, or combinations thereof; and programming a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

Example 2: The method of example 1, further including at least one of delivering, according to the determined electrical stimulation settings, the subsequent electrical stimulation to the patient to evoke the one or more subsequent stimulation-evoked signals from the patient, or sensing, according to the determined sensing settings, the one or more subsequent stimulation-evoked signals from the patient in response to the subsequent electrical stimulation.

Example 3: The method of example 1 or example 2, further including delivering therapeutic electrical stimulation based on the one or more subsequent stimulation-evoked signals.

Example 4: The method of any one of examples 1-3, wherein the one or more different signal types comprise at least one of an evoked neural signal type, an electromyography (EMG) signal type, or a composite evoked neural signal type signal type and EMG signal type.

Example 5: The method of any one of examples 1-4, wherein the characteristics comprise one or more signal features comprising at least one of a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve feature, a signal maximum rate of change, or a signal minimum rate of change.

Example 6: The method of any one of examples 1-5, wherein the electrical stimulation settings comprise settings for delivering subsequent electrical stimulation configured to at least one of: evoke an ECAP signal, evoke an EMG signal, evoke a composite ECAP and EMG signal, evoke an ECAP signal followed by an EMG signal, or evoke an EMG signal followed by an ECAP signal.

Example 7: The method of example 6, wherein determining characteristics of the one or more different signal types comprises: sweeping through a delivery of a plurality of electrical stimulation pulses, wherein each electrical stimulation pulse is characterized by at least an electrode configuration, a pulse width, a polarity, a frequency, an amplitude, a recharge parameter, or a pulse pattern; and sweeping through sensing parameters to sense one or more stimulation-evoked signals, wherein the one or more sensing parameters comprises an electrode configuration or a blanking parameter.

Example 8: The method of example 7, wherein each sensed stimulation-evoked signal of the one or more different signal types is sensed after a corresponding electrical stimulation pulse or a set of corresponding stimulation pulses of the plurality of electrical stimulation pulses.

Example 9: The method of example 7 or example 8, wherein the plurality of electrode configurations comprises at least one of a monopolar configuration, a bipolar configuration, or a tripolar configuration, and wherein the plurality of pulses comprises a pulse that is at least 20 microseconds and less than or equal to 450 microseconds.

Example 10: The method of any one of examples 1-9, wherein the electrical stimulation settings for delivering subsequent electrical stimulation to a patient for evoking at least one of the signal types comprises a masker-probe pulse sequence.

Example 11: The method of any one of examples 1-10, wherein the electrical stimulation settings for delivering subsequent electrical stimulation to a patient for evoking at least one of the signal types comprises alternating the polarity of consecutive pulses.

Example 12: The method of any one of examples 1-11, wherein the characteristics are first characteristics, wherein the electrical stimulation settings are first electrical stimulation settings, wherein the sensing settings are first sensing settings, the method further including determining, for the patient, second characteristics of one or more different signal types that are received in response to using at least two settings selected from the group of one or more electrical stimulation settings, one or more sensing settings, or combinations thereof; programming the neurostimulation system, based on the second characteristics, to include at least one of: second electrical stimulation settings for delivering subsequent electrical stimulation to a patient for evoking at least one of the signal types, or second sensing settings for sensing at least one of the signal types in response to stimulation of the patient; and at least one of delivering, according to the determined second electrical stimulation settings, the subsequent electrical stimulation to the patient to evoke the one or more subsequent stimulation-evoked signals from the patient, or sensing, according to the second signal sensing settings, the one or more subsequent stimulation-evoked signals from the patient in response to the subsequent electrical stimulation.

Example 13: A system including: at least one electrode configured to deliver the electrical stimulation to a patient; and a device including processing circuitry configured to: determine, for a patient, characteristics of one or more different signal types that are received in response to using at least two settings selected from the group of one or more electrical stimulation settings, one or more sensing settings, or combinations thereof; and program a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

Example 14: The system of example 13, wherein the processing circuitry is further configured to: at least one of deliver, according to the determined electrical stimulation settings, the subsequent electrical stimulation to the patient to evoke the one or more subsequent stimulation-evoked signals from the patient, or sense, according to the determined sensing settings, the one or more subsequent stimulation-evoked signals from the patient in response to the subsequent electrical stimulation.

Example 15: The system of example 13 or example 14, wherein the processing circuitry is further configured to deliver therapeutic electrical stimulation based on the one or more subsequent stimulation-evoked signals.

Example 16: The system of any one of examples 13-15, wherein the one or more different signal types comprise at least one of an evoked neural signal type, an electromyography (EMG) signal type, or a composite evoked neural signal type signal type and EMG signal type.

Example 17: The system of any one of examples 13-16, wherein the characteristics comprise one or more signal features comprising at least one of a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve feature, a signal maximum rate of change, or a signal minimum rate of change.

Example 18: The system of any one of examples 13-17, wherein the electrical stimulation settings comprise settings for delivering subsequent electrical stimulation configured to at least one of: evoke an ECAP signal, evoke an EMG signal, evoke a composite ECAP and EMG signal, evoke an ECAP signal followed by an EMG signal, or evoke an EMG signal followed by an ECAP signal.

Example 19: The system of example 18, wherein the processing circuitry is configured to determine characteristics of the two or more different signal types by: sweeping through a delivery of a plurality of electrical stimulation pulses, wherein each electrical stimulation pulse is characterized by at least an electrode configuration, a pulse width, a polarity, a frequency, an amplitude, a recharge parameter, or a pulse pattern; and sweeping through sensing parameters to sense one or more stimulation-evoked signals, wherein the one or more sensing parameters comprises an electrode configuration or a blanking parameter.

Example 20: The system of example 19, wherein each sensed stimulation-evoked signal of the two or more different signal types is sensed after a corresponding electrical stimulation pulse or a set of corresponding stimulation pulses of the plurality of electrical stimulation pulses.

Example 21: The system of example 19 or example 20, wherein the plurality of electrode configurations comprises at least one of a monopolar configuration, a bipolar configuration, or a tripolar configuration, and wherein the plurality of pulses comprises a pulse that is at least 20 microseconds and less than or equal to 450 microseconds.

Example 22: The system of any one of examples 13-21, wherein the electrical stimulation settings for delivering subsequent electrical stimulation to a patient for evoking at least one of the signal types comprises a masker-probe pulse sequence.

Example 23: The system of any one of examples 13-22, wherein the electrical stimulation settings for delivering subsequent electrical stimulation to a patient for evoking at least one of the signal types comprises alternating the polarity of consecutive pulses.

Example 24: The system of any one of examples 13-23, wherein the characteristics are first characteristics, wherein the electrical stimulation settings are first electrical stimulation settings, wherein the sensing settings are first sensing settings, wherein the processing circuitry is further configured to: determine, for the patient, second characteristics of two or more different signal types that are received in response to using at least two settings selected from the group of one or more electrical stimulation settings, one or more sensing settings, or combinations thereof; program the neurostimulation system, based on the second characteristics, to include at least one of: second electrical stimulation settings for delivering subsequent electrical stimulation to a patient for evoking at least one of the signal types, or second sensing settings for sensing at least one of the signal types in response to stimulation of the patient; and at least one of deliver, according to the determined second electrical stimulation settings, the subsequent electrical stimulation to the patient to evoke the one or more subsequent stimulation-evoked signals from the patient, or sense, according to the second signal sensing settings, the one or more subsequent stimulation-evoked signals from the patient in response to the subsequent electrical stimulation.

Example 25: A computer readable medium including instructions that when executed cause one or more processors to: determine, for a patient, characteristics of one or more different signal types that are received in response to using at least two settings selected from the group of one or more electrical stimulation settings, one or more sensing settings, or combinations thereof; and program a neurostimulation system, based on the characteristics, at least one of: electrical stimulation settings for delivering subsequent electrical stimulation to a patient for evoking at least one of the signal types, or sensing settings for sensing at least one of the signal types in response to stimulation of the patient.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

I/We claim:

1. A system comprising:
at least one electrode configured to deliver the electrical stimulation to a patient; and
a device comprising processing circuitry configured to:
determine, for a patient, characteristics of one or more different signal types that are received in response to using at least two settings selected from the group of one or more electrical stimulation settings, one or more sensing settings, or combinations thereof; and
program a neurostimulation system, based on the characteristics, at least one of:
electrical stimulation settings for delivering subsequent electrical stimulation to a patient for evoking at least one of the signal types, or
sensing settings for sensing at least one of the signal types in response to stimulation of the patient.

2. The system of claim 1, wherein the processing circuitry is further configured to:
at least one of deliver, according to the determined electrical stimulation settings, the subsequent electrical stimulation to the patient to evoke the one or more subsequent stimulation-evoked signals from the patient, or sense, according to the determined sensing settings, the one or more subsequent stimulation-evoked signals from the patient in response to the subsequent electrical stimulation.

3. The system of claim 1, wherein the processing circuitry is further configured to deliver therapeutic electrical stimulation based on the one or more subsequent stimulation-evoked signals.

4. The system of claim 1, wherein the one or more different signal types comprise at least one of an evoked neural signal type, an electromyography (EMG) signal type, or a composite evoked neural signal type signal type and EMG signal type.

5. The system of claim 1, wherein the characteristics comprise one or more signal features comprising at least one of a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve feature, a signal maximum rate of change, or a signal minimum rate of change.

6. The system claim 1, wherein the electrical stimulation settings comprise settings for delivering subsequent electrical stimulation configured to at least one of:
evoke an ECAP signal,
evoke an EMG signal,
evoke a composite ECAP and EMG signal,
evoke an ECAP signal followed by an EMG signal, or
evoke an EMG signal followed by an ECAP signal.

7. The system of claim 6, wherein the processing circuitry is configured to determine characteristics of the two or more different signal types by:
sweeping through a delivery of a plurality of electrical stimulation pulses, wherein each electrical stimulation pulse is characterized by at least an electrode configuration, a pulse width, a polarity, a frequency, an amplitude, a recharge parameter, or a pulse pattern; and
sweeping through sensing parameters to sense one or more stimulation-evoked signals, wherein the one or more sensing parameters comprises an electrode configuration or a blanking parameter.

8. The system of claim 7, wherein each sensed stimulation-evoked signal of the two or more different signal types is sensed after a corresponding electrical stimulation pulse or a set of corresponding stimulation pulses of the plurality of electrical stimulation pulses.

9. The system of claim 7, wherein the plurality of electrode configurations comprises at least one of a monopolar configuration, a bipolar configuration, or a tripolar configuration, and
wherein the plurality of pulses comprises a pulse that is at least 20 microseconds and less than or equal to 450 microseconds.

10. The system of claim 1, wherein the electrical stimulation settings for delivering subsequent electrical stimulation to a patient for evoking at least one of the signal types comprises a masker-probe pulse sequence.

11. The system of claim 2, wherein the electrical stimulation settings for delivering subsequent electrical stimulation to a patient for evoking at least one of the signal types comprises alternating the polarity of consecutive pulses.

12. The system of claim 2, wherein the characteristics are first characteristics, wherein the electrical stimulation settings are first electrical stimulation settings, wherein the sensing settings are first sensing settings, wherein the processing circuitry is further configured to:
determine, for the patient, second characteristics of two or more different signal types that are received in response to using at least two settings selected from the group of one or more electrical stimulation settings, one or more sensing settings, or combinations thereof;
program the neurostimulation system, based on the second characteristics, to include at least one of:
second electrical stimulation settings for delivering subsequent electrical stimulation to a patient for evoking at least one of the signal types, or
second sensing settings for sensing at least one of the signal types in response to stimulation of the patient; and
at least one of deliver, according to the determined second electrical stimulation settings, the subsequent electrical stimulation to the patient to evoke the one or more subsequent stimulation-evoked signals from the patient, or sense, according to the second signal sensing settings, the one or more subsequent stimulation-evoked signals from the patient in response to the subsequent electrical stimulation.

13. A computer readable medium comprising instructions that when executed cause one or more processors to:
determine, for a patient, characteristics of one or more different signal types that are received in response to using at least two settings selected from the group of one or more electrical stimulation settings, one or more sensing settings, or combinations thereof; and
program a neurostimulation system, based on the characteristics, at least one of:
electrical stimulation settings for delivering subsequent electrical stimulation to a patient for evoking at least one of the signal types, or
sensing settings for sensing at least one of the signal types in response to stimulation of the patient.
